(19) United States
(12) Patent Application Publication
(10) Pub. No.: US 2015/0146862 A1
(43) Pub. Date: May 28, 2015

(54) X-RAY IMAGING APPARATUS AND CONTROL METHOD THEREOF

(71) Applicant: Samsung Electronics Co., Ltd., Suwon-si (KR)

(72) Inventor: Myeong Je KIM, Seoul (KR)

(73) Assignee: Samsung Electronics Co., Ltd., Suwon-si (KR)

(21) Appl. No.: 14/555,764

(22) Filed: Nov. 28, 2014

(30) Foreign Application Priority Data
Nov. 28, 2013 (KR) 10-2013-0145963

(51) Int. Cl. A61B 6/00; G01T 1/17

(52) U.S. Cl. CPC A61B 6/4494; G01T 1/17
USPC 378/91

(57) ABSTRACT

An X-ray imaging apparatus includes an X-ray source to generate and irradiate X-rays; an X-ray detector installed in at least one module of one or more modules, to detect the irradiated X-rays; an Identification (ID) resistor included in the module; a port included in the X-ray detector; and a controller to determine a module in which the X-ray detector has been installed, using the ID resistor and the port. The controller may assign a static Information Provider (IP) address to the module, and determine a module in which the X-ray detector has been installed, based on the static IP address. According to the X-ray imaging apparatuses and a control method thereof, it is possible to determine an installation location of the X-ray detector, and to use the X-ray detector in various modules without having to provide separate X-ray detectors for different locations.

### CROSS-REFERENCE TO RELATED APPLICATIONS

This application claims the priority benefit of Korean Patent Application No. 10-2013-0145963, filed on Nov. 28, 2013 in the Korean Intellectual Property Office, the disclosure of which is incorporated herein by reference.

### BACKGROUND

1. Field

The following description relates to an X-ray imaging apparatus of irradiating X-rays onto an object to produce an X-ray image, and a control method of the X-ray imaging apparatus.

2. Description of the Related Art

An X-ray imaging apparatus is equipment for acquiring images of the inside of an object using X-rays. The X-ray imaging apparatus images the inside of an object using a non-invasive method of irradiating X-rays onto the object and detecting X-rays transmitted through the object. Accordingly, a medical X-ray imaging apparatus is used to diagnose an internal injury or a disease of an object that cannot be examined externally.

The X-ray imaging apparatus includes an X-ray source to generate X-rays and to irradiate the X-rays onto an object, and an X-ray detector to detect X-rays transmitted through the object. In order to image various parts of an object, the X-ray source is configured to be movable, and the X-ray detector is configured to be installable in a radiography table, a radiography stand, and a radiography portable.

Lately, with digitalization of X-ray imaging apparatuses, X-ray images are acquired digitally instead of on film so that many functions of X-ray imaging apparatuses are automated. Examples of such automation are Auto Tracking of an X-ray source, automatically tracking an X-ray detector, and Auto Centering of automatically centering the locations of the X-ray source and the X-ray detector. In order to implement automation functions of an X-ray imaging apparatus, such as Auto Tracking or Auto Centering, it is necessary to correctly distinguish between X-ray detectors installed in a radiography table, a radiography stand, and a radiography portable.

### SUMMARY

Therefore, it is an aspect of the present disclosure to provide an X-ray imaging apparatus capable of determining a location at which an X-ray detector has been installed, and distinguishing between X-ray detectors installed in a radiography table, a radiography stand, and a radiography portable, and a control method of the X-ray imaging apparatus.

Additional aspects of the disclosure will be set forth in part in the description which follows and, in part, will be obvious from the description, or may be learned by practice of the disclosure.

In accordance with an aspect of the present disclosure, an X-ray imaging apparatus may include an X-ray source configured to generate and irradiate X-rays; an X-ray detector installed in at least one module of one or more modules, and configured to detect the irradiated X-rays; an Identification (ID) resistor included in the module; a port included in the X-ray detector; and a controller configured to determine a module in which the X-ray detector has been installed, using the ID resistor and the port.

The module may include at least one radiography table.

The module may include at least one radiography stand.

The module may include at least one radiography portable.

The module may include a power box connected to the controller and a power supply unit.

The ID resistor may be included in the power box.

A power box of the module in which the X-ray detector has been installed may be connected to the X-ray detector so that power is supplied to the X-ray detector and the X-ray detector is able to communicate with the controller.

The port may include an Analog-Digital Converter (ADC) port.

ID resistors having different resistance values may be respectively included in different power boxes.

The port may monitor a voltage in correspondence to the ID resistor of the power box connected to the X-ray detector, and output a digital value corresponding to a level of the voltage to the controller.

The controller may determine the module in which the X-ray detector has been installed, based on the digital value output from the port.

The port may include a General Purpose Input/Output (GPIO) port.

The port may be at least one port included in the X-ray detector.

The ID resistor may at least one ID resistor included in the power box in correspondence to the number of the at least one port.

The at least one port may one-to-one match the at least one ID resistor included in the power box.

The at least one ID resistor may have the same resistance value.

Each of the at least one ID resistor may be a pull-up resistor or a pull-down resistor.

Different ordered pairs of ID resistors may be respectively included in different power boxes.

Each of the at least one port may monitor a voltage in correspondence to an ID resistor matching the corresponding port among at least one ID resistor of a power box to which the X-ray detector has been connected, and output a relative level of the voltage to the controller.

The controller may determine the module in which the X-ray detector has been installed, based on the relative level of the voltage output from the port.

In accordance with an aspect of the present disclosure, an X-ray imaging apparatus may include an X-ray source configured to generate and irradiate X-rays; an X-ray detector installed in at least one module of one or more modules, and configured to detect the irradiated X-rays; and a controller configured to assign a fixed Information Provider (IP) address to the module, and to determine a module in which the X-ray detector has been installed, based on the fixed IP address.

Each module may include a power box connected to the controller and a power supply unit.

The fixed IP address may be assigned to the power box.

A power box of the module in which the X-ray detector has been installed may be connected to the X-ray detector so that power is supplied to the X-ray detector and the X-ray detector is able to communicate with the controller.

The controller may receive a fixed IP address of a power box to which the X-ray detector is connected.

The controller may determine the module in which the X-ray detector has been installed, based on the fixed IP address.

The controller may maintain or change an IP address assigned to the X-ray detector in correspondence to the module in which the X-ray detector has been installed.

The controller may move the X-ray source to correspond to a location of the X-ray detector.

In accordance with an aspect of the present disclosure, a control method of an X-ray imaging apparatus may include, at a port included in an X-ray detector, monitoring a voltage in correspondence to an Identification (ID) resistor of a module in which the X-ray detector has been installed; at the port, outputting a data value corresponding to the voltage; and determining the module in which the X-ray detector has been installed, based on the data value.

In accordance with an aspect of the present disclosure, a control method of an X-ray imaging apparatus may include assigning a fixed Information Provider (IP) address to at least one module; receiving a fixed IP address of a module in which an X-ray detector has been installed; and determining the module in which an X-ray detector has been installed, based on the received fixed IP address.

The control method of an X-ray imaging apparatus may further include maintaining or changing an IP address assigned to the X-ray detector in correspondence to the module in which the X-ray detector has been installed.

The control method of an X-ray imaging apparatus may further include moving the X-ray source to correspond to a location of the X-ray detector.

According to the X-ray imaging apparatuses and the control methods thereof, it is possible to determine an installation location of the X-ray detector, to detect the X-ray detector installed in any one of the radiography table, the radiography stand, and the radiography portable, and to use the X-ray detector in various modules without having to provide separate X-ray detectors for the radiography table, for the radiography stand, and for the radiography portable.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a perspective view of an X-ray imaging apparatus according to an embodiment of the present disclosure;

FIG. 2 is an exploded perspective view of an X-ray imaging apparatus according to an embodiment of the present disclosure;

FIG. 3 is a front view of an operating unit of an X-ray imaging apparatus, according to an embodiment of the present disclosure;

FIG. 4 is a control block diagram of an X-ray imaging apparatus according to an embodiment of the present disclosure;

FIG. 5 illustrates an example of an internal structure of an X-ray tube;

FIG. 6 schematically illustrates a structure of a sensing panel;

FIG. 7 is a circuit diagram schematically illustrating a pixel area of the sensing panel illustrated in FIG. 6;

FIG. 8 is a view for describing a method in which an X-ray detector is installed in a radiography table;

FIG. 9 is a view for describing a method in which an X-ray detector is installed in a radiography stand;

FIG. 10 is a view for describing a method in which an X-ray detector is installed in a radiography portable;

FIG. 11 illustrates an X-ray detector connected to a power box;

FIG. 12 is a view for describing an example of a method of determining an installation location of an X-ray detector based on the control block diagram of FIG. 4;

FIG. 13 is a view for describing a method of assigning an Information Provider (IP) address to an X-ray detector and a method of changing an IP address of the X-ray detector;

FIGS. 14, 15, and 16 are views for describing movement of an X-ray source in an automatic move mode;

FIG. 17 is a control block diagram of an X-ray imaging apparatus according to an embodiment of the present disclosure;

FIG. 18 is a view for describing an example of a method of determining an installation location of an X-ray detector based on the control block diagram of FIG. 17;

FIG. 19 is a view for describing an example of a method of determining an installation location of an X-ray detector based on the control block diagram of FIG. 17;

FIG. 20 is a view for describing a method of assigning an IP address to an X-ray detector and a method of changing an IP address of the X-ray detector;

FIG. 21 is a control block diagram of an X-ray imaging apparatus according to still an embodiment of the present disclosure;

FIG. 22 is a view for describing an example of a method of determining an installation location of an X-ray detector based on the control block diagram of FIG. 21;

FIG. 23 is a flowchart illustrating a control method of an X-ray imaging apparatus, according to an embodiment of the present disclosure;

FIG. 24 is a flowchart illustrating a control method of an X-ray imaging apparatus, according to an embodiment of the present disclosure; and

FIG. 25 is a flowchart illustrating a control method of an X-ray imaging apparatus, according to embodiment of the present disclosure.

### DETAILED DESCRIPTION

Reference will now be made in detail to the embodiments of the present disclosure, examples of which are illustrated in the accompanying drawings, wherein like reference numerals refer to like elements throughout.

Hereinafter, an X-ray imaging apparatus and a control method thereof according to embodiments of the present disclosure will be described in detail with reference to the accompanying drawings.

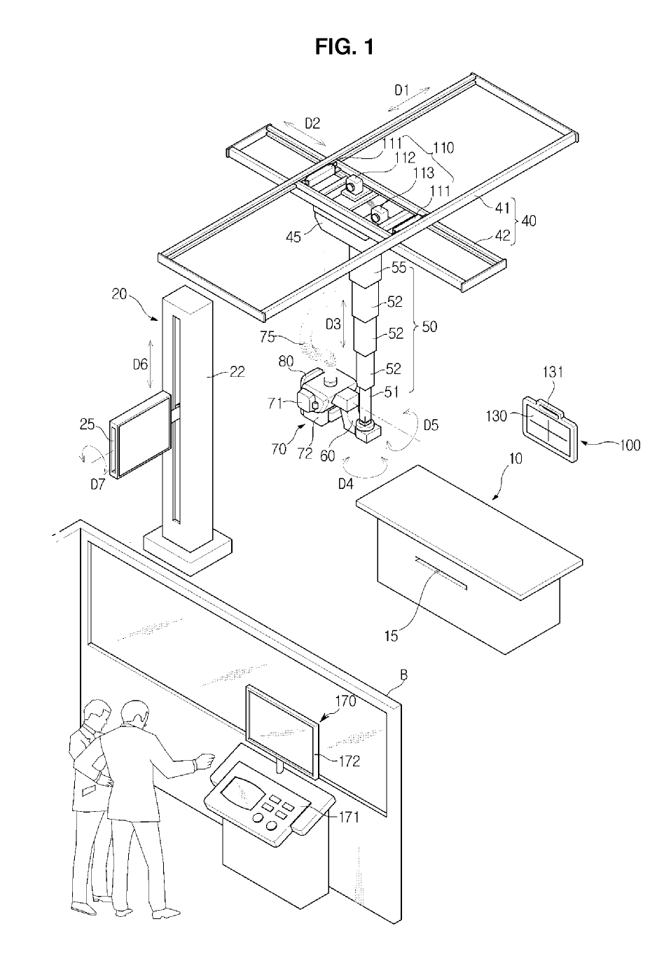

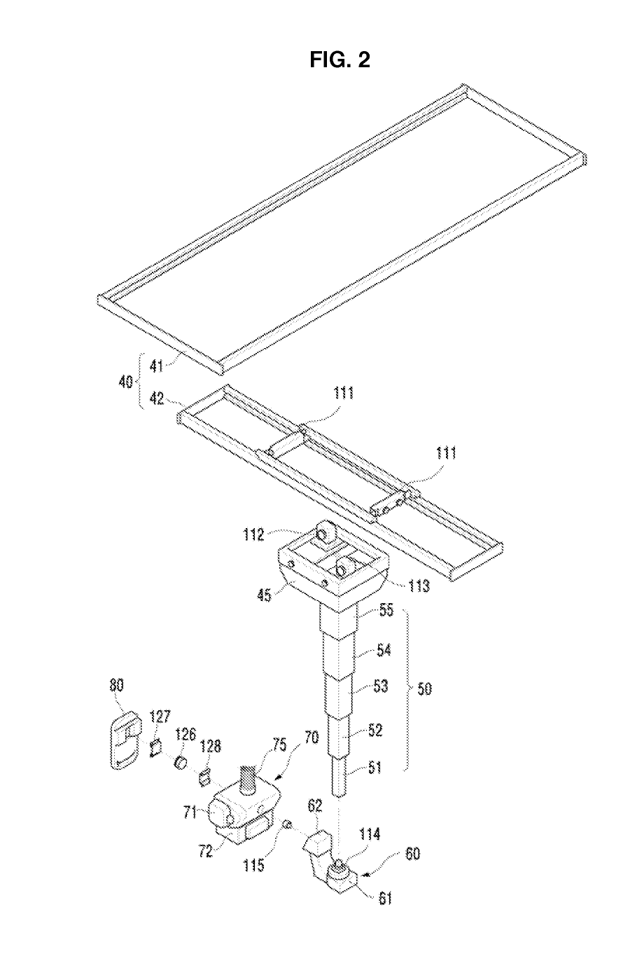

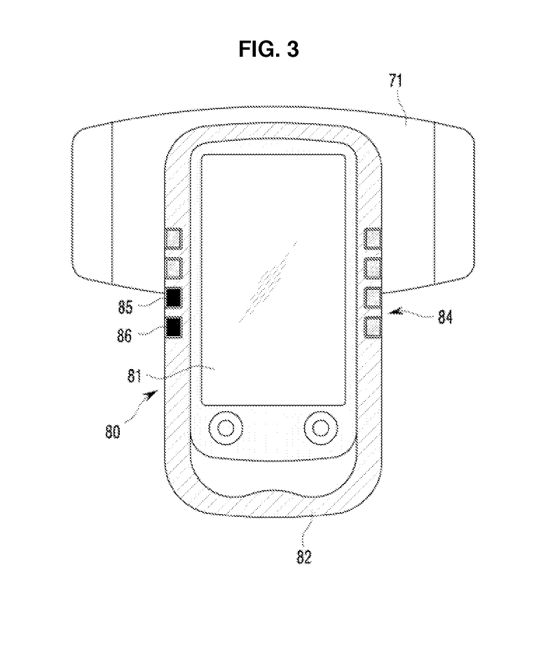

FIG. 1 is a perspective view of an X-ray imaging apparatus according to an embodiment of the present disclosure, FIG. 2 is an exploded perspective view of an X-ray imaging apparatus according to an embodiment of the present disclosure, and FIG. 3 is a front view of an operating unit of an X-ray imaging apparatus, according to an embodiment of the present disclosure.

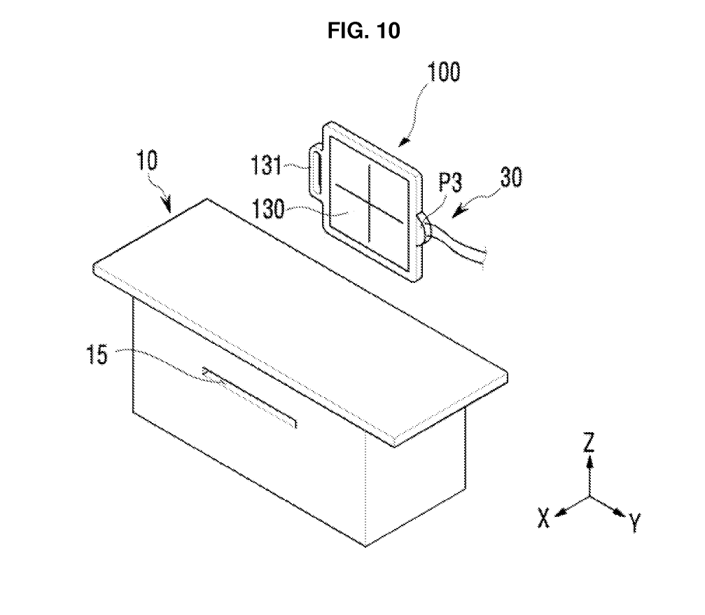

Referring to FIGS. 1, 2, and 3, an X-ray imaging apparatus 1 may include a guide rail unit 40, a carriage 45, a post frame 50, a motor unit 110, an X-ray source 70, an X-ray detector 100, an operating unit 80, and a workstation 170. The X-ray imaging apparatus 1 may further include a radiography table 10, a radiography stand 20, and a radiography portable 30 (see FIG. 10) in which the X-ray detector 100 can be installed.

The guide rail unit 40, the carriage 45, and the post frame 50 are used to move the X-ray source 70 toward an object.

The guide rail unit 40 may include a first guide rail 41 and a second guide rail 42 arranged to form a predetermined angle with respect to each other. The first guide rail 41 may be orthogonal to the second guide rail 42.

The first guide rail 41 may be installed on a ceiling of an examination room where a radiography apparatus is placed.

The second guide rail 42 may be disposed beneath the first guide rail 41, and slide with respect to the first guide rail 41. The first guide rail 41 may include a plurality of rollers (not shown) that are movable along the first guide rail 41. The second guide rail 42 may connect to the rollers and move along the first guide rail 41.

A direction in which the first guide rail 41 extends is defined as a first direction D1, and a direction in which the second guide rail 42 extends is defined as a second direction D2. Accordingly, the first direction D1 may be orthogonal to the second direction D2, and the first and second directions D1 and D2 may be parallel to the ceiling of the examination room.

The carriage 45 may be disposed beneath the second guide rail 42, and move along the second guide rail 42. The carriage 45 may include a plurality of rollers (not shown) to move along the second guide rail 42. Accordingly, the carriage 45 is movable in the first direction D1 together with the second guide rail 42, and movable in the second direction D2 along the second guide rail 42. The post frame 50 may be fixed on the carriage 45 and disposed below the carriage 45. The post frame 50 may include a plurality of posts 51, 52, 53, 54, and 55.

The posts 51, 52, 53, 54, and 55 may connect to each other such that they can be folded with each other. The length of the post frame 50 fixed on the carriage 45 may increase or decrease in the elevation direction of the examination room.

A direction in which the length of the post frame 50 increases or decreases is defined as a third direction D3. Accordingly, the third direction D3 may be orthogonal to the first direction D1 and the second direction D2.

The X-ray source 70 may irradiate X-rays to an object. Herein, the object may be a human's or animal's living body, however, the object is not limited to these. That is, the object may be anything whose inside structure can be imaged by the X-ray imaging apparatus 1.

The X-ray source 70 may include an X-ray tube 71 to generate X-rays, and a collimator 72 to guide the generated X-rays to be headed toward an object. The X-ray tube 71 will be described in more detail, later.

A revolute joint 60 may be disposed between the X-ray source 70 and the post frame 50.

The revolute joint 60 may couple the X-ray source 70 with the post frame 50, and support a load applied to the X-ray source 70.

The revolute joint 60 may include a first revolute joint 61 connected to the lower post 51 of the post frame 50, and a second revolute joint 62 connected to the X-ray source 70.

The first revolute joint 61 is rotatable with respect to the central axis of the post frame 50 extending in the elevation direction of the examination room. Accordingly, the first revolute joint 61 may rotate on a plane that is perpendicular to the third direction D3. The rotation direction of the first revolute joint 61 is defined as a fourth direction D4, and the fourth direction D4 is a rotation direction of an axis parallel to the third direction D3.

The second revolute joint 62 is rotatable on a plane that is perpendicular to the ceiling of the examination room. Accordingly, the second revolute joint 62 may rotate in a rotation direction of an axis parallel to the first direction D1 and the second direction D2. The rotation direction of the second rotation joint 62 is defined as a fifth direction D5, and the fifth direction D5 is a rotation direction of an axis extending in the first direction D1 or the second direction D2. The X-ray source 70 may connect to the revolute joint 60 and rotate in the fourth direction D4 and the third direction D5. Also, the X-ray source 70 may connect to the post frame 50 through the revolute joint 60, and linearly move in the first direction D1, in the second direction D2, or in the third direction D3.

In order to move the X-ray source 70 in the first direction D1 through the fifth direction D5, the motor unit 110 is used. The motor unit 110 may be electrically driven, and may include encoders.

The motor unit 110 may include a first motor 111, a second motor 112, a third motor 113, a fourth motor 114, and a fifth motor 115 that correspond to the first direction D1, the second direction D2, the third direction D3, the fourth direction D4, and the fifth direction D5, respectively.

The first to fifth motors 111 to 115 may be arranged at appropriate locations in consideration of convenience of design. For example, the first motor 111 that is used to move the second guide rail 42 in the first direction D1 may be disposed around the first guide rail 41, the second motor 112 that is used to move the carriage 45 in the second direction D2 may be disposed around the second guide rail 42, and the third motor 113 that is used to increase or decrease the length of the post frame 50 in the third direction D3 may be disposed in the carriage 45. Also, the fourth motor 114 that is used to rotate the X-ray source 70 in the fourth direction D4 may be disposed around the first revolute joint 61, and the fifth motor 115 that is used to rotate the X-ray source 70 in the fifth direction D5 may be disposed around the second revolute joint 62.

The motor unit 110 may connect to a power transfer (not shown) in order to linearly move or rotate the X-ray source 70 in the first to fifth directions D1 to D5. The power transfer may be a belt and a pulley, a chain and a sprocket, or a shaft, for example.

In one side of the X-ray source 70, the operating unit 80 may be disposed to provide a user interface. The user is a person who diagnoses an object using the X-ray imaging apparatus 1, and may be a medical staff including a doctor, a radiological technologist, and a nurse. However, the user is not limited to the above-mentioned persons, and may be anyone using the X-ray imaging apparatus 1. The operating unit 80 may be connected to the X-ray source 70 by connectors 126, 127, and 128. The operating unit may receive network and power connections through connector 75.

The operating unit 80 may include, as illustrated in FIG. 3, a first display unit 81 and a plurality of buttons 84 to allow a user to input various kinds of information for radiography or to manipulate individual units. The first display unit 81 may be implemented as a Cathode Ray Tube (CRT), a Liquid Crystal Display (LCD), or a Light Emitting Diode (LED) display, for example. However, the first display unit 81 is not limited to the above-mentioned display devices.

The buttons 84 may include a fourth directional rotation selection button 85 and a fifth directional rotation selection button 86 to rotate the X-ray source 70 in the fourth direction D4 and in the fifth direction D5. That is, when a user wants to rotate the X-ray source 70 in the fourth direction D4, the user may rotate the X-ray source 70 in the fourth direction D4 after pressing the fourth directional rotation selection button 85 or while pressing the fourth directional rotation selection button 85. When the user wants to rotate the X-ray source 70 in the fifth direction D5, the user may rotate the X-ray source 70 in the fifth direction D5 after pressing the fifth directional rotation selection button 86 or while pressing the fifth directional rotation selection button 86. The locations of the fourth and fifth directional rotation selection buttons 85 and 86 shown in FIG. 3 are exemplary, and the fourth and fifth directional rotation selection buttons 85 and 86 may be arranged at different locations.

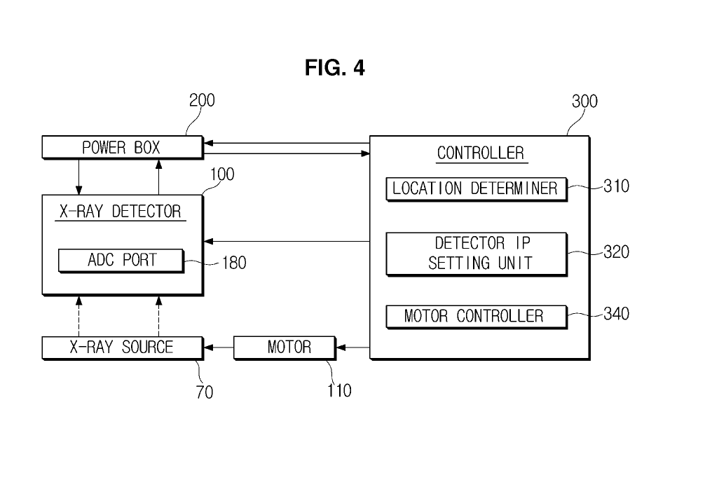

Also, the operating unit 80 may include a handle 82 that the user can grip. The user may grip the handle 82 of the operating unit 80 to apply power or torque, thereby moving the X-ray source 70. This is defined as a manual move mode, and an automatic move mode will be defined when a motor controller 340 (see FIG. 4) is described. In FIG. 3, the handle 82 is provided in the lower part of the operating unit 80, however, the handle 82 may be provided at another location.

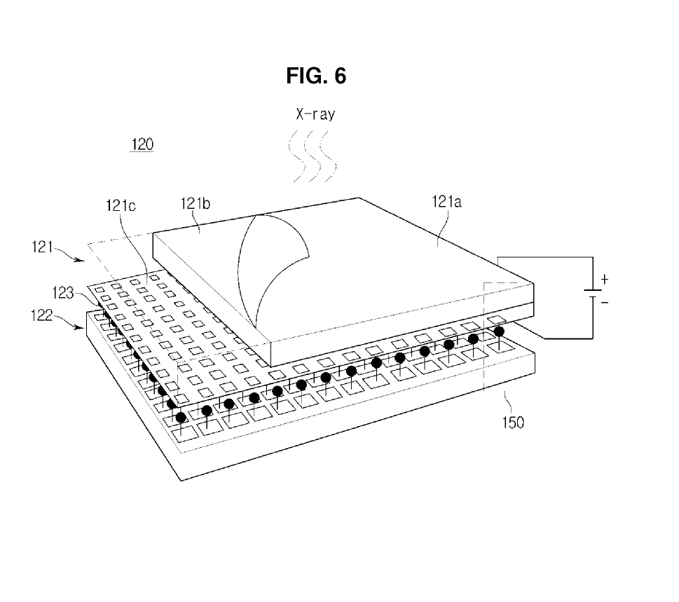

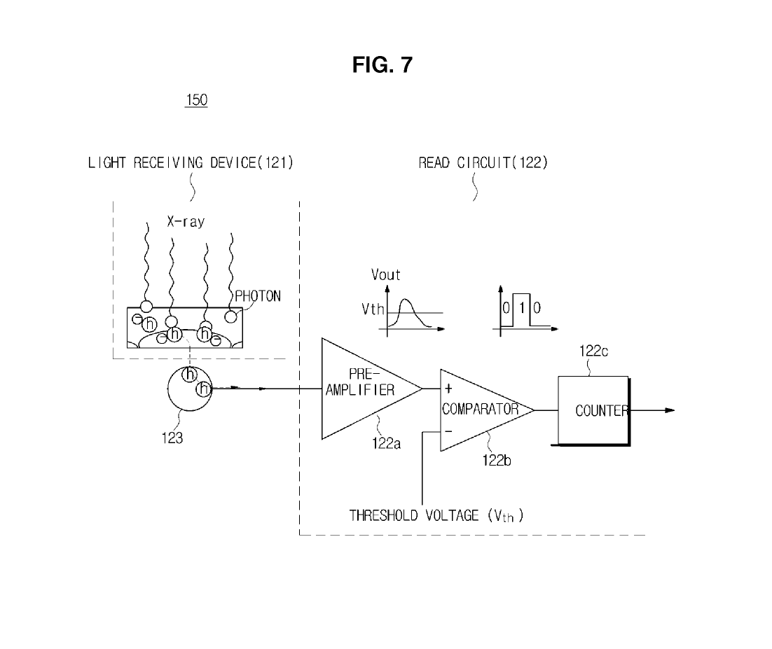

The X-ray detector 100 may detect X-rays transmitted through the object. In the front side of the X-ray detector 100, an incident surface 130 onto which X-rays are incident may be provided, and a sensing panel 120 (see FIG. 6) may be installed in the X-ray detector 100. In the sensing panel 120, a plurality of pixels 150 (see FIG. 7) that respond to X-rays may be arranged in a matrix form, which will be described in more detail with reference to FIG. 6, later. In the upper center part of the X-ray detector 100, a handle 131 may be provided so that the user can move or carry the X-ray detector 100.

The X-ray detector 100 may be installed in a radiography table 10, a radiography stand 20, or a radiography portable 30 (see FIG. 10) when radiography is performed. Particularly, accommodating slots 15 and 25 into which the X-ray detector 100 can be inserted may be formed in the radiography table 10 and in the radiography stand 20. The accommodating slot 15 formed in the radiography table 10 is defined as a first accommodating slot 15, and the accommodating slot 25 formed in the radiography stand 20 is defined as a second accommodating slot 25. The second accommodating slot 25 is movable in the length direction of a support bar 22, and rotatable in the rotation direction of an axis perpendicular to the length direction of the support bar 22, as illustrated in FIG. 1. The length direction of the support bar 22 is defined as a sixth direction D6, and the rotation direction of the axis perpendicular to the sixth direction D6 is defined as a seventh direction D7. A method of installing the X-ray detector 100 will be described in detail, later.

The workstation 170 may include an input unit 171 and a second display unit 172 to provide a user interface, like the operating unit 80. Accordingly, the user can input various kinds of information for radiography or manipulate individual units through the workstation 170. Also, the user may input various commands (e.g., a command for selecting a radiography location, a start command for radiography, etc.) related to operations of the X-ray imaging apparatus 1 through the workstation 170. In addition, the user may check images acquired during radiography through the workstation 170.

The input unit 171 may include at least one of a switch, a keyboard, a trackball, a mouse, and a touch screen, for example. If the input unit 171 is implemented as a Graphic User Interface (GUI) such as a touch screen, in other words, if the input unit 171 is implemented in software, the input unit 171 may be displayed through the second display unit 172. The second display unit 172 may be, like the first display unit 81, implemented as a CRT, a LCD, or a LED display, for example.

The workstation 170 may include various processors, such as a Central Processing Unit (CPU) or a Graphic Processing Unit (GPU), and a Printed Circuit Board (PCB), and may further include various kinds of storage units as necessary. Accordingly, the workstation 170 may accommodate main components (e.g., the controller 300 (see FIG. 4)) of the X-ray imaging apparatus 1 to make determinations for operations of the X-ray imaging apparatus 1 or to generate various control signals.

The workstation 170 may be placed in an independent space B from which X-rays can be blocked, and may be connected to the X-ray source 70, the X-ray detector 100, the radiography table 10, the radiography stand 20, and the radiography portable 30 through wired/wireless communication.

FIG. 4 is a control block diagram of the X-ray imaging apparatus 1 according to an embodiment of the present disclosure.

Referring to FIG. 4, the X-ray imaging apparatus 1 may determine a location of the X-ray detector 100 using the X-ray source 70, the X-ray detector 100, a power box unit 200, a controller 300, and the motor unit 110, and move the X-ray source 70 to correspond to the location of the X-ray detector 100.

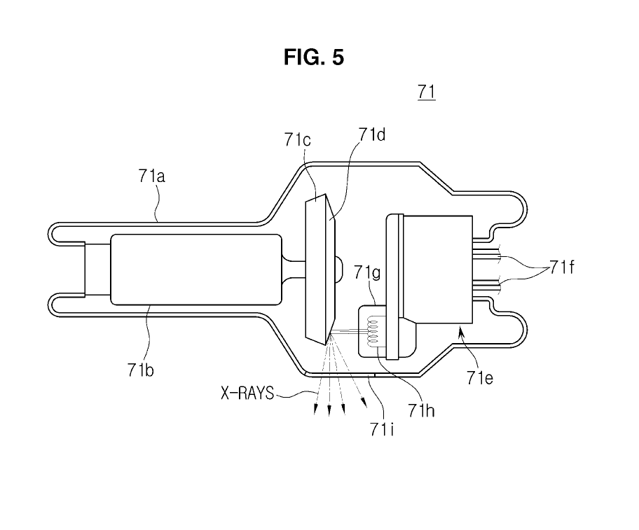

The X-ray source 70 may generate X-rays, and irradiate the generated X-rays to an object. In order to generate X-rays, the X-ray source 70 may include an X-ray tube 71 as illustrated in FIG. 5. FIG. 5 illustrates an example of an internal structure of the X-ray tube 71.

The X-ray tube 71 may be embodied as a two-electrode vacuum tube including an anode 71c and a cathode 71e. The body of the two-electrode vacuum tube may be a glass tube 71a made of silica (hard) glass or the like.

The cathode 71e may include a filament 71h and a focusing electrode 71g for focusing electrons, and the focusing electrode 71g is also called a focusing cup. The inside of the glass tube 71a may be evacuated to a high vacuum state of approximately 10 mmHg, and the filament 71h of the cathode 71e may be heated to a high temperature, thereby generating thermoelectrons. The filament 71h may be a tungsten filament, for example, and the filament 71h may be heated by applying a current to electrical leads 71f connected to the filament 71h. However, instead of the filament 71h, a carbon nano-tube capable of being driven with high-speed pulses may be used as the cathode 71e.

The anode 71c may be made of copper, for example, and a target material 71d is applied on the surface of the anode 71c facing the cathode 71e, wherein the target material 71d may be a high-resistance material, e.g., Cr, Fe, Co, Ni, W, or Mo. The higher the melting point of the target material 71d, the smaller the focal spot size.

When a high voltage is applied between the cathode 71e and the anode 71c, thermoelectrons may be accelerated and collide with the target material 71d of the anode 71e, thereby generating X-rays. The X-rays may be irradiated to the outside through a window 71i. The window 111i may be a Beryllium (Be) thin film.

The target material 71d may be rotated by a rotor 71b. When the target material 71d rotates, the heat accumulation rate may increase 10 times per unit area and the focal spot size may be reduced, compared to when the target material 71d is fixed.

The voltage that is applied between the cathode 71e and the anode 71c of the X-ray tube 71 is called a tube voltage. The magnitude of a tube voltage may be expressed as a crest value (kVp). When the tube voltage increases, a velocity of thermoelectrons may increase accordingly. Then, energy (energy of photons) of X-rays that are generated when the thermoelectrons collide with the target material 71d may also increase. A current flowing through the X-ray tube 71 is called a tube current, and can be expressed as an average value (mA). When a tube current increases, a dose of X-rays (that is, the number of X-ray photons) may increase. In summary, an energy level of X-rays can be controlled by adjusting a tube voltage. Also, a dose of X-rays can be controlled by adjusting a tube current and an X-ray exposure time.

The X-ray detector 100 may detect X-rays irradiated by the X-ray source 70 and then transmitted through an object. The X-rays may be detected by the sensing panel 120 installed in the X-ray detector 100. The sensing panel 120 may convert the detected X-rays into electrical signals, and acquire an image about the inside of the object.

The sensing panel 120 can be classified according to its material configuration, a method of converting detected X-rays into electrical signals, and a method of acquiring image signals.

The sensing panel 120 is classified into a mono type device or a hybrid type device according to its material configuration.

If the sensing panel 120 is a mono type device, a part of detecting X-rays and generating electrical signals, and a part of reading and processing the electrical signals may be semiconductors made of the same material, or may be manufactured by one process. In this case, the sensing panel 120 may be a Charge Coupled Device (CCD) or a Complementary Metal Oxide Semiconductor (CMOS) which is a light receiving device.

If the sensing panel 120 is a hybrid type device, a part of detecting X-rays and generating electrical signals, and a part of reading and processing the electrical signals may be made of different materials, or may be manufactured by different processes. For example, there are cases of detecting X-rays using a light receiving device, such as a photodiode, a CCD, or CdZnTe, and reading and processing electrical signals using a CMOS Read Out Integrated Circuit (CMOS ROIC), of detecting X-rays using a strip detector, and reading and processing electrical signals using a CMOS ROIC, and of using an a-Si or a-Se flat panel system.

The X-ray detector 120 may use a direct conversion mode and an indirect conversion mode according to a method of converting X-rays into electrical signals.

In the direct conversion mode, if X-rays are irradiated, electron-hole pairs are temporarily generated in a light receiving device, electrons move to an anode, and holes move to a cathode by an electric field applied to both terminals of the light receiving device. The sensing panel 120 converts the movements of the electrons and holes into electrical signals. The light receiving device may be made of a-Se, CdZnTe, HgI2, or PbI2, for example.

In the indirect conversion mode, if X-rays irradiated from the X-ray source 70 react with a scintillator to emit photons having a wavelength of a visible light region, the light receiving device detects the photons, and converts the photons into electrical signals. The light receiving device may be made of a-Si, and the scintillator may be a GADOX scintillator of a thin film type, or a CSI (TI) of a micro pillar type or a needle type.

The sensing panel 120 may use a Charge Integration Mode (CIM) of storing charges for a predetermined time period and then acquiring a signal from the stored charges, or a Photon Counting Mode (PCM) of counting the number of photons having energy higher than threshold energy whenever a signal is generated by single X-ray photons, according to a method of acquiring image signals.

The material configuration of the sensing panel 120 and the signal conversion method of the sensing panel 120 are not limited, however, for convenience of description, in an embodiment of the present disclosure which will be described below, the sensing panel 120 uses the direct conversion mode of acquiring electrical signals directly from X-rays and the PCM, and the sensing panel 120 is a hybrid type in which a sensor chip for detecting X-rays is integrated with a read circuit chip.

The sensing panel 120 may have a 2-dimensional (2D) pixel array structure including a plurality of pixels 150, as illustrated in FIG. 6. FIG. 6 schematically illustrates a structure of the sensing panel 120.

Referring to FIG. 6, the sensing panel 120 may include a light receiving device 121 to detect X-rays and convert the X-rays into electrical signals, and a read circuit 122 to read out the electrical signals.

The light receiving device 121 may be made of a single crystal semiconductor material in order to ensure high resolution, high response speed, and a high dynamic area even under conditions of low energy and a small dose of X-rays. The single crystal semiconductor material may be Ge, CdTe, CdZnTe, or GaAs.

The light receiving device 121 may be in the form of a PIN photodiode. The PIN photodiode may be fabricated by bonding a p-type semiconductor substrate 121c in the form of a 2D pixel array on the lower surface of a n-type semiconductor substrate 121b having high resistance.

The read circuit 122, which is fabricated according to a Complementary Metal Oxide Semiconductor (CMOS) process, may form a 2D array structure, and may be coupled with the p-type substrate 121c of the light receiving device 121 in units of pixels. The CMOS read circuit 122 and the light receiving device 121 may be coupled by a Flip-Chip Bonding (FCB) method. More specifically, the CMOS read circuit 122 and the light receiving device 121 may be coupled by forming bumps 123 with PbSn, In, or the like, reflowing, applying heat, and then compressing.

FIG. 7 is a circuit diagram schematically illustrating a pixel area of the sensing panel 120 illustrated in FIG. 6.

Referring to FIG. 7, if photons of X-rays are incident to the light receiving device 121, electrons existing in a valance band may receive the energy of the photons to be excited to a conduction band over an energy gap of a band gap. Thereby, electron-hole pairs may be generated in a depletion region where neither electrons nor holes exist.

If a reverse bias is applied after metal electrodes are respectively formed on the p-type layer and the n-type substrate of the light receiving device 121, electrons in the electron-hole pairs generated in the depletion region may move to the n-type region, and holes in the electron-hole pairs may move to the p-type region. The holes moved to the p-type region may be input to the read circuit 122 through the bumps 123.

Charges input to the read circuit 122 may be transferred to a pre-amplifier 122a, and the pre-amplifier 122a may output a voltage signal corresponding to the charges.

The voltage signal output from the pre-amplifier 122a may be transferred to a comparator 122b. The comparator 122b may compare the voltage signal to a predetermined threshold voltage that can be controlled by an external device, to output a pulse signal of “1” or “0” as the result of the comparison. More specifically, if a voltage of the voltage signal is greater than the predetermined threshold voltage, the comparator 122b may output a signal of “1”, and if the voltage of the voltage signal is smaller than the predetermined threshold voltage, the comparator 122b may output a signal of “0”. The counter 122c may count the number of times a signal of “1” has been generated, and output the count value as digital data.

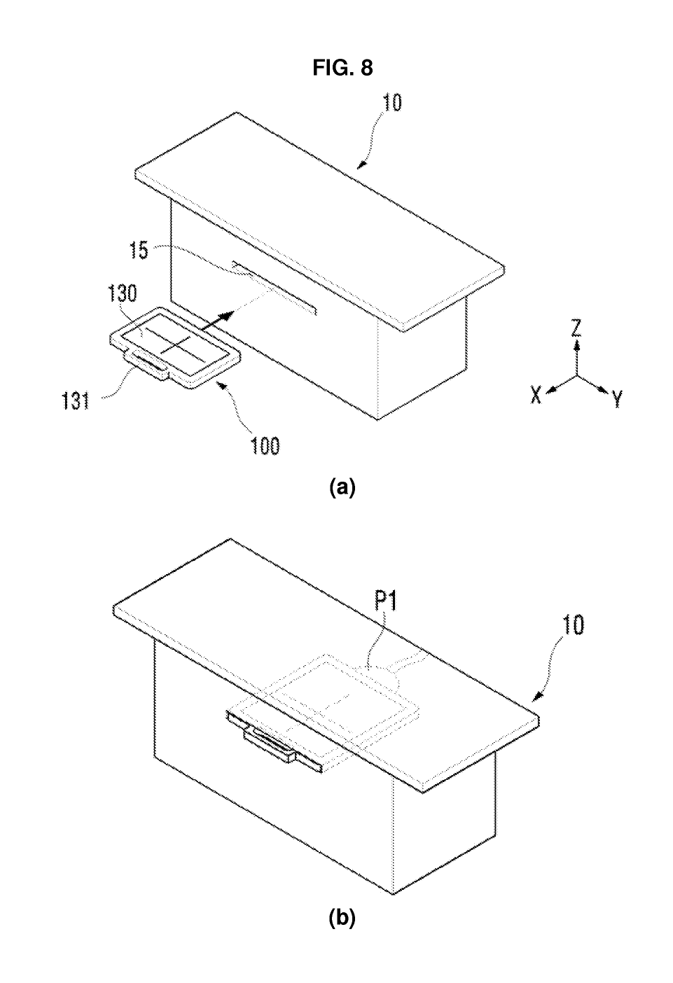

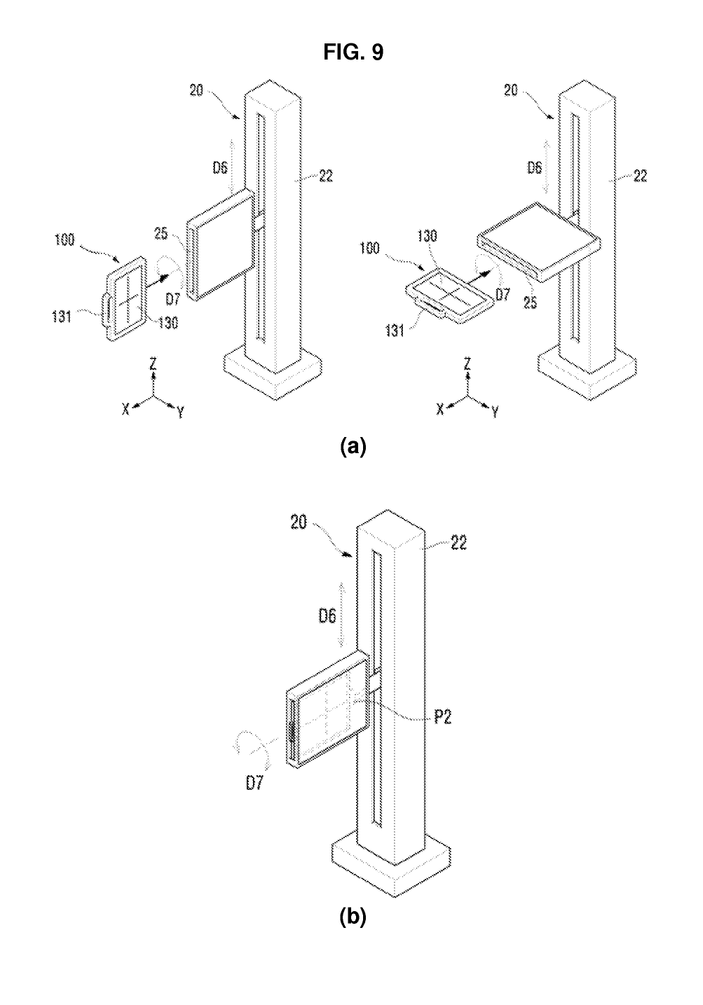

As described above, the X-ray detector 100 is installed in the radiography table 10, the radiography stand 20, or the radiography portable 30 in order to detect X-rays, and a method in which the X-ray detector 100 is installed in each module will be described with reference to FIGS. 8, 9, and 10, below. FIG. 8 is a view for describing a method in which the X-ray detector 100 is installed in the radiography table 10, FIG. 9 is a view for describing a method in which the X-ray detector 100 is installed in the radiography stand 20, and FIG. 10 is a view for describing a method in which the X-ray detector 100 is installed in the radiography portable 30.

In order to perform radiography on an object that is lying on the radiography table 10, the X-ray detector 100 may be installed in the radiography table 10. More specifically, the X-ray detector 100 may be inserted into the first accommodating slot 15 formed in the radiography table 10. When the X-ray detector 100 is inserted into the first accommodating slot 15, the X-ray detector 100 is inserted in a state of being parallel to a bottom plane, that is, a plane formed by x- and y-axes, as illustrated in FIG. 8(a). After the X-ray detector 100 is inserted into the first accommodating slot 15, the X-ray detector 100 may be maintained in the state of being parallel to the bottom plane or the plane formed by x- and y-axes, as illustrated in FIG. 8(b). Meanwhile, the X-ray detector 100 inserted into the first accommodating slot 15 may be connected to a first power box P1 included in the radiography table 10. As such, a state in which the X-ray detector 100 has been inserted into the first accommodating slot 15 and connected to the first power box P1 is a state in which the X-ray detector 100 has been installed in the radiography table 10.

In order to perform radiography on an object that stands in front of the radiography stand 20, the X-ray detector 100 may be installed in the radiography stand 20. More specifically, the X-ray detector 100 may be inserted into the second accommodating slot 25 formed in the radiography stand 20. Because the second accommodating slot 25 is rotatable in the seventh direction D7, the X-ray detector 100 may be inserted into the second accommodating slot 25 in a state of being perpendicular to a bottom plane or parallel to a plane formed by x- and z-axes, as illustrated in the left side of FIG. 9(a), or the X-ray detector 100 may be inserted into the second accommodating slot 25 in a state of being parallel to the bottom plane or parallel to a plane formed by x- and y-axes, as illustrated in the right side of FIG. 9(a). After the X-ray detector 100 is inserted into the second accommodating slot 25, the second accommodating slot 25 may rotate so that the X-ray detector 100 is maintained in a state of being perpendicular to the bottom plane, that is, parallel to the plane formed by x- and z-axes, as illustrated in FIG. 9(b). Meanwhile, the X-ray detector 100 inserted into the second accommodating slot 25 may be connected to a second power box P2 included in the radiography stand 20. As such, a state in which the X-ray detector 100 has been inserted into the second accommodating slot 25 and connected to the second power box P2 is a state in which the X-ray detector 100 has been installed in the radiography stand 20.

In order to perform radiography on a moving object as well as an object that is lying or stands, the X-ray detector 100 may be installed in the radiography portable 30. The radiography portable 30 includes a third power box P3, and a cable connected to one end of the third power box P3. Accordingly, a state in which the X-ray detector 100 has been connected to the third power box P3 is a state in which the X-ray detector 100 has been installed in the radiography portable 30. The X-ray detector 100 may be installed in the radiography portable 30, and movable together with the radiography portable 30. The X-ray detector 100 may be inserted in the underside of the top plate of the radiography table 10, as illustrated in FIG. 10.

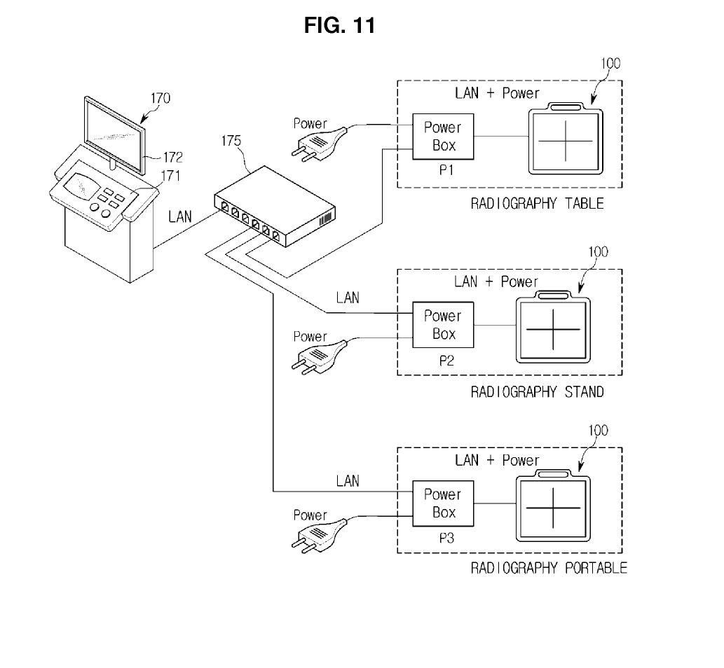

The X-ray detector 100 installed in the radiography table 10, the radiography stand 20, or the radiography portable 30 may connect to a power supply unit and the workstation 170 through the power box unit 200. In other words, the power box unit 200 may include the first power box P1 included in the radiography table 10, the second power box P2 included in the radiography stand 20, and the third power box P3 included in the radiography portable 30. The power box unit 200 may supply power to the X-ray detector 100 installed in the radiography table 10, the radiography stand 20, or the radiograph portable 30, and connect the X-ray detector 100 to the workstation 170. The connection relationship between the power box unit 200 and the X-ray detector 100 will be described in more detail with reference to FIG. 11, below. FIG. 11 illustrates the X-ray detector 100 connected to the power box unit 200.

If the X-ray detector 100 is installed in the radiography table 10, the X-ray detector 100 may be connected to the first power box P1. The first power box P1 may connect to a power supply unit, and supply power transferred from the power supply unit to the X-ray detector 100 connected to the first power box P1. Also, the first power box P1 may connect to the workstation 170 through a network hub 175 and a cable. Accordingly, the first power box P1 may output various command signals received from the workstation 170 to the X-ray detector 100 connected to the first power box P1, and output various data received from the X-ray detector 100 connected to the first power box P1 to the workstation 170.

If the X-ray detector 100 is installed in the radiography stand 20, the X-ray detector 100 may be connected to the second power box P2. The second power box P2 may connect to a power supply unit, and connect to the workstation 170 through the network hub 175 and a cable. Accordingly, the second power box P2 may supply power transferred from the power supply unit to the X-ray detector 100 connected to the second power box P2. Also, the second power box P2 may output various command signals received from the workstation 170 to the X-ray detector 100 connected to the second power box P2, and output various data received from the X-ray detector 100 connected to the second power box P2 to the workstation 170.

Likewise, if the X-ray detector 100 is installed in the radiography portable 30, the X-ray detector 100 may be connected to the third power box P3. The third power box P3 may connect to a power supply unit, and supply power transferred from the power supply unit to the X-ray detector 100 connected to the third power box P3. Also, the third power box P3 may connect to the workstation 170 through the network hub 175 and a cable to output various command signals received from the workstation 170 to the X-ray detector 100 connected to the third power box P3 and to output various data received from the X-ray detector 100 connected to the third power box P3 to the workstation 170.

The X-ray detector 100 may be connected to the first, second, and third power boxes P1, P2, and P3 through communication lines of Local Area Network (LAN). Also, cables connecting the workstation 170 to the power boxes P1, P2, and P3, more specifically, a cable connecting the workstation 170 to the network hub 175 and cables connecting the network hub 175 to the power boxes P1, P2, and P3 may include the communication lines of LAN, as illustrated in FIG. 11.

Referring again to FIG. 4, the controller 300 may include a location determiner 310, a detector IP setting unit 320, and a motor controller 340.

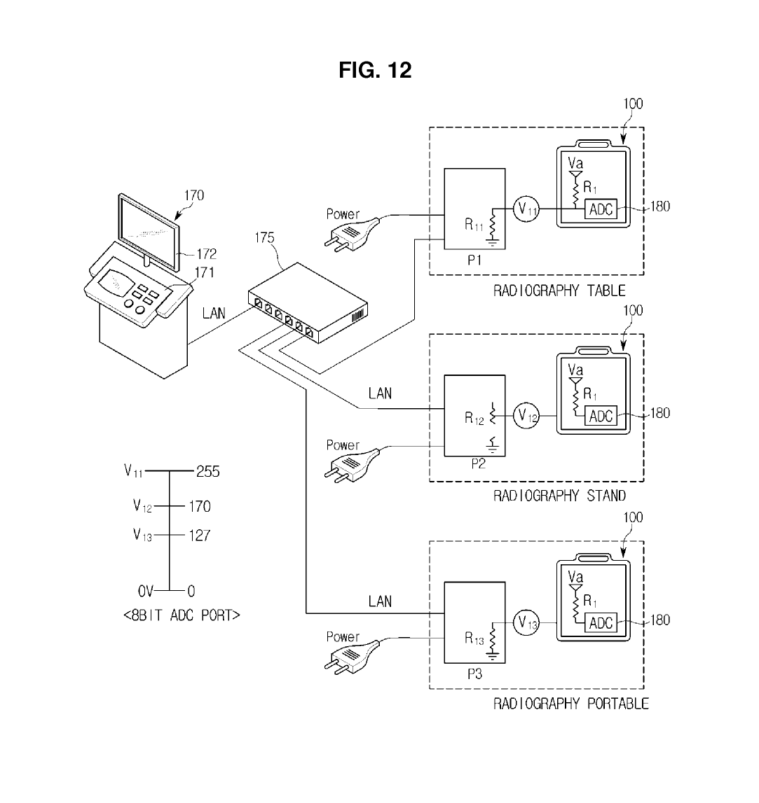

The location determiner 310 may determine a location at which the X-ray detector 100 has been installed. In order to help a determination by the location determiner 310, the X-ray detector 100 may include a port (e.g., an Analog-Digital Converter (ADC) port) for monitoring a voltage, and the power box unit 200 may include a unique Identification (ID) resistor. A method in which the location determiner 310 determines an installation location of the X-ray detector 100 using the port and the ID resistor will be described in detail with reference to FIG. 12, below.

FIG. 12 is a view for describing an example of a method of determining an installation location of the X-ray detector 100 based on the control block diagram of FIG. 4.

The X-ray detector 100 may include a port for monitoring a voltage and converting a level of the monitored voltage into a digital value. In FIG. 12, as an example of the port, an ADC port 180 is shown. The first power box P1 includes an ID resistor R11, the second power box P2 includes an ID resistor R12, and the third power box P3 includes an ID resistor R13. The ID resistors R11, R12, and R13 have different resistance values.

A voltage Va is applied to the X-ray detector 100. The voltage Va may be applied through a voltage probe. Because the ID resistors R11, R12, and R13 included in the respective power boxes P1, P2, and P3 have different resistance values, a voltage that is monitored by the ADC port 180 varies depending on which module the X-ray detector 100 has been installed in.

In detail, if the X-ray detector 100 has been installed in the radiography table 10, the ADC port 180 may monitor a voltage V11 applied to an ID check line in correspondence to the ID resistor R11 included in the first power box P1. If the X-ray detector 100 has been installed in the radiography stand 20, the ADC port 180 may monitor a voltage V12 applied to an ID check line in correspondence to the ID resistor R12 included in the second power box P2. If the X-ray detector 100 has been installed in the radiography portable 30, the ADC port 180 may monitor a voltage V13 applied to an ID check line in correspondence to the ID resistor R13 included in the third power box P3.

The ADC port 180 may convert the monitored voltage into a digital value corresponding to a level of the monitored voltage, and output the digital value to the location determiner 310. As described above, because a voltage that is monitored by the ADC port 180 varies depending on which module the X-ray detector 100 has been installed in, a value which the ADC port 180 outputs to the location determiner 310 also varies depending on which module the X-ray detector 100 has been installed in. In the current example, it is assumed that the ADC port 180 is an 8 bit ADC. That is, it is assumed that the ADC port 180 can output a value ranging from 0 to 255.

If the X-ray detector 100 has been installed in the radiography table 10 and monitored a voltage V11, for example, a voltage of 5V, the ADC port 180 may output a digital value (e.g., 255) corresponding to 5V to the location determiner 310. If the X-ray detector 100 has been installed in the radiography stand 20 and monitored a voltage V12, for example, a voltage of 3.2V, the ADC port 180 may output a digital value (e.g., 170) corresponding to 3.2V to the location determiner 310. If the X-ray detector 100 has been installed in the radiography portable 30 and monitored a voltage V13, for example, a voltage of 2.5V, the ADC port 180 may output a digital value (e.g., 127) corresponding to 2.5V to the location determiner 310.

Because the voltage Va that is applied to the X-ray detector 100 is constant, and a resistance value of an ID resistor also does not change in a module, the ADC port 180 may output a constant digital value when the X-ray detector 100 has been installed in a module. If the X-ray detector 100 has been installed in the radiography table 10, the ADC port 180 outputs a value of 255, and if the X-ray detector 100 has been installed in the radiography stand 20, the ADC port 180 outputs a value of 170. Also, if the X-ray detector 100 has been installed in the radiography portable 30, the ADC port 180 outputs a value of 127.

Accordingly, the location determiner 310 may determine a location at which the X-ray detector 100 has been installed, based on a digital value output from the ADC port 180. If the ADC port 180 has output a value of 255, the location determiner 310 may determine that the X-ray detector 100 has been installed in the radiography table 10, and if the ADC port 180 has output a value of 170, the location determiner 310 may determine that the X-ray detector 100 has been installed in the radiography stand 20. Also, if the ADC port 180 has output a value of 127, the location determiner 310 may determine that the X-ray detector 100 has been installed in the radiography portable 30.

For convenience of description, a case in which there are a radiography table 10, a radiography stand 20, and a radiography portable 30 has been described. However, the number of modules is not limited. In other words, both the radiography table 10 and the radiography stand 20 may be provided, or a plurality of radiography tables, such as a first radiography table and a second radiography table, and/or a plurality of radiography stands, such as a first radiography stand and a second radiography stand, may be provided.

When a plurality of radiography tables 10 are provided, the radiography tables 10 may be considered as different modules. Likewise, when a plurality of radiography stands 20 or a plurality of radiography portables 30 are provided, the radiography stands 20 or the radiography portables 30 may also be considered as different modules. For example, if a first radiography table, a second radiography table, a first radiography stand, a second radiography stand, and a first radiography portable are provided, the first radiography table, the second radiography table, the first radiography stand, the second radiography stand, and the first radiography portable may be considered as different modules.

Power boxes included in different modules include different ID resistors. Accordingly, the number of digital values that can be output from the ADC port 180 corresponds to the number of different modules. For example, if a first radiography table, a second radiography table, a first radiography stand, a second radiography stand, and a first radiography portable are provided, the number of digital values that can be output from the ADC port 180 is five in correspondence to the number of the modules. In other words, a value that is output from the ADC port 180 when the X-ray detector 100 has been installed in the first radiography table, a value that is output from the ADC port 180 when the X-ray detector 100 has been installed in the second radiography table, a value that is output from the ADC port 180 when the X-ray detector 100 has been installed in the first radiography stand, a value that is output from the ADC port 180 when the X-ray detector 100 has been installed in the second radiography stand, and a value that is output from the ADC port 180 when the X-ray detector 100 has been installed in the first radiography portable are different values. When the X-ray detector 100 has been installed in a predetermined module, the ADC port 180 outputs a constant value corresponding to the module.

Even when there are more or less modules than those shown in FIG. 12, the location determiner 310 can determine an installation location of the X-ray detector 100 based on a digital value output from the ADC port 180.

In the current example, the ADC port 180 is an 8 bit ADC. However, the ADC port 180 may be a 4 bit ADC, a 10 bit ADC, a 12 bit ADC, or a 14 bit ADC according to the number of modules.

The detector IP setting unit 320 (see FIG. 4) may set IP addresses in correspondence to the number of modules, and assign an IP address corresponding to an installation location of the X-ray detector 100 to the X-ray detector 100, or change an IP address assigned to the X-ray detector 100 according to an installation location of the X-ray detector 100.

First, the detector IP setting unit 320 may set IP addresses in correspondence with the number of modules. If there are a plurality of radiography tables 10, a plurality of radiography stands 20, and/or a plurality of radiography portables 30, for example, if there are a first radiography table, a second radiography table, a first radiography stand, a second radiography stand, and a first radiography portable, the detector IP setting unit 320 may set an IP address for the first radiography table, an IP address for the second radiography table, an IP address for the first radiography stand, an IP address for the second radiography stand, and an IP address for the first radiography portable to different values in correspondence with the number of the modules.

If there is a radiography table 10, a radiography stand 20, and a radiography portable 30, the detector IP setting unit 320 may set an IP address for the radiography table 10, an IP address for the radiography stand 20, and an IP address for the radiography portable 30 to different values in correspondence to the number of the modules. For example, the detector IP setting unit 320 may set an IP address for the radiography table 10 to 192.168.10.1, an IP address for the radiography stand 20 to 192.168.0.2, and an IP address for the radiography portable 30 to 192.168.0.3.

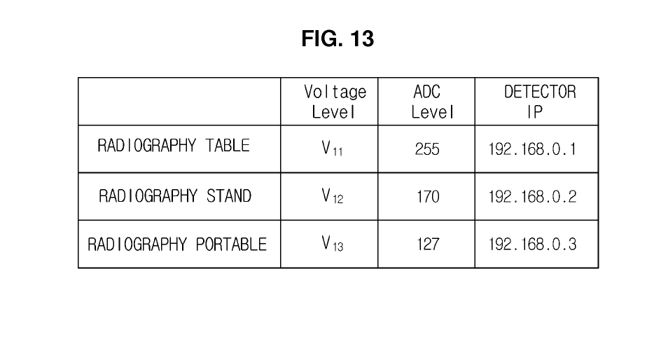

Then, the detector IP setting unit 320 may assign an IP address to the X-ray detector 100 or change an IP address of the X-ray detector 100, based on a determination of the location determiner 310. This will be described in more detail with reference to FIG. 13, below. FIG. 13 is a view for describing a method of assigning an IP address to the X-ray detector 100 and a method of changing an IP address of the X-ray detector 100. The following description will be given based on the example of FIG. 12.

As described above with reference to FIG. 12, if the ADC port 180 has monitored a voltage V11 and output a value of 255 corresponding to a level of the voltage V11, the location determiner 310 may determine that the X-ray detector 100 has been installed in the radiography table 10. The detector IP setting unit 320 may assign the IP address 192.168.0.1 for the radiography table 10 to the X-ray detector 100 based on the determination of the location determiner 310. If an IP address that is different from 192.168.0.1 has already been assigned to the X-ray detector 100, the detector IP setting unit 320 may change the IP address of the X-ray detector 100 to 192.168.0.1 so that the IP address of the X-ray detector 100 is identical to the IP address for the radiography table 10.

If the ADC port 180 has monitored a voltage V12 and output a value of 170 corresponding to a level of the voltage V12, the location determiner 310 may determine that the X-ray detector 100 has been installed in the radiography stand 20. Accordingly, the detector IP setting unit 320 may assign the IP address 192.168.0.2 for the radiography stand 20 to the X-ray detector 100. If an IP address that is different from 192.168.0.2 has already been assigned to the X-ray detector 100, the detector IP setting unit 320 may change the IP address of the X-ray detector 100 to 192.168.0.2 so that the IP address of the X-ray detector 100 is identical to the IP address for the radiography stand 20.

If the ADC port 180 has monitored a voltage V13 and output a value of 127 corresponding to a level of the voltage V13, the location determiner 310 may determine that the X-ray detector 100 has been installed in the radiography portable 30. Accordingly, the detector IP setting unit 320 may assign the IP address 192.168.0.3 for the radiography portable 30 to the X-ray detector 100. If an IP address that is different from 192.168.0.3 has already been assigned to the X-ray detector 100, the detector IP setting unit 320 may change the IP address of the X-ray detector 100 to 192.168.0.3 so that the IP address of the X-ray detector 100 is identical to the IP address for the radiography portable 30.

As such, because the detector IP setting unit 320 assigns an IP address to the X-ray detector 100 or changes an IP address of the X-ray detector 100 according to an installation location of the X-ray detector 100, the X-ray detector 100 can be used at various locations. For example, the X-ray detector 100 can be used in any one of the first radiography table, the second radiography table, the first radiography stand, the second radiography stand, and the first radiography portable, and the X-ray detector 100 can be used in any one of the radiography table 10, the radiography stand 20, and the radiography portable 30.

Meanwhile, the user may apply power or torque to the handle 82 of the operating unit 80 (see FIG. 3), based on the determination of the location determiner 310 so as to move the X-ray source 70 to correspond to the location of the X-ray detector 100. This is the manual move mode defined above.

The automatic move mode can be defined in correspondence to the manual move mode. In the automatic move mode, the motor controller 340 controls driving of the motor unit 110 in order to move the X-ray source 70.

More specifically, if a user sets a radiography mode based on a determination of the location determiner 310, the motor controller 340 may detect locations of the X-ray source 70 and the X-ray detector 100, and output a control signal(s) to a motor(s) that needs to be driven. The X-ray source 70 may move to correspond to the location of the X-ray detector 100 according to driving of the motor unit 110. The user's setting may be done through the first display unit 81, the buttons 84, or the input unit 171 or the second display unit 172 of the workstation 170. A mode of moving the X-ray source 70 according to a user's setting is defined as the automatic move mode.

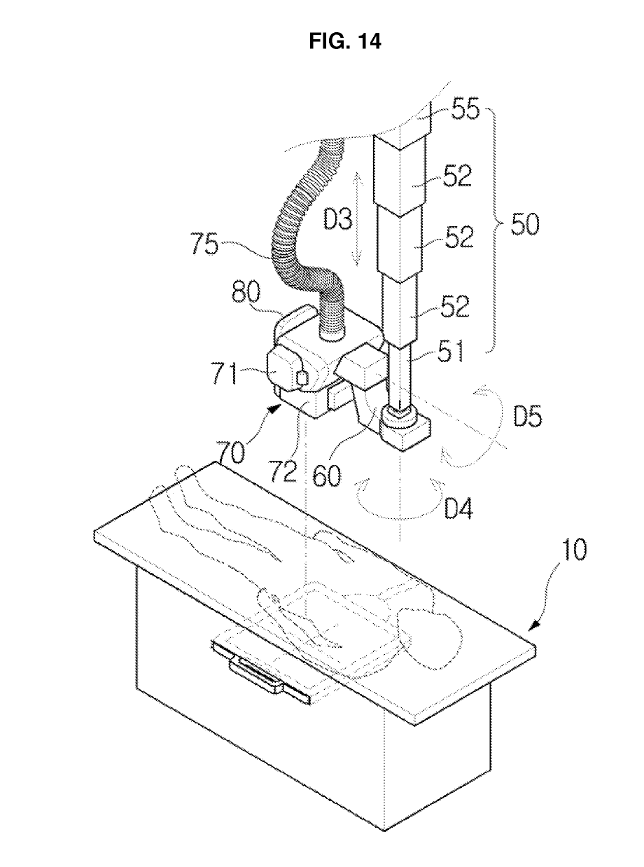

FIGS. 14, 15, and 16 are views for describing movement of the X-ray source 70 in the automatic move mode. In FIGS. 14, 15, and 16, a case in which there is a radiography table 10, a case in which there is a radiography stand 20, and a case in which there is a radiography portable 30 are respectively shown.

If the location determiner 310 determines that the X-ray detector 100 has been installed in the radiography table 10, a user may set a radiography mode to “table”. According to the user's setting, the motor controller 340 (see FIG. 4) may output a control signal for controlling driving of the motor unit 110 in order to move the X-ray source 70 to the radiography table 10. If the motor unit 110 is driven according to the control signal, the X-ray source 10 moves to the location of the X-ray detector 100 installed in the radiography table 10, as illustrated in FIG. 14. In this state, X-rays irradiated from the X-ray source 70 can be detected by the X-ray detector 100 installed in the radiography table 10.

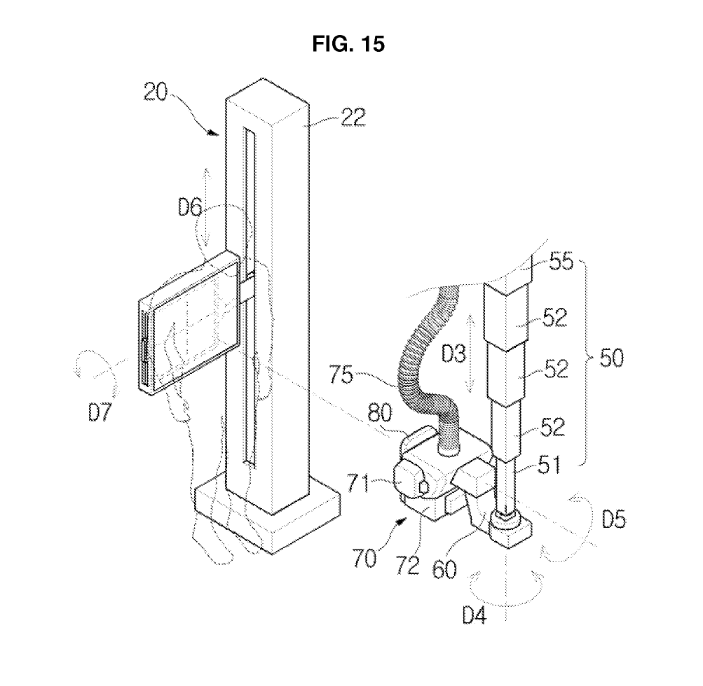

If the location determiner 310 determines that the X-ray detector 100 has been installed in the radiography stand 20, the user may set a radiography mode to “stand”. According to the user's setting, the motor controller 340 may output a control signal for controlling driving of the motor unit 110 in order to move the X-ray source 70 to the radiography stand 20. If the motor unit 110 is driven according to the control signal, the X-ray source 10 moves to the location of the X-ray detector 100 installed in the radiography stand 20, as illustrated in FIG. 15. In this state, X-rays irradiated from the X-ray source 70 can be detected by the X-ray detector 100 installed in the radiography stand 20.

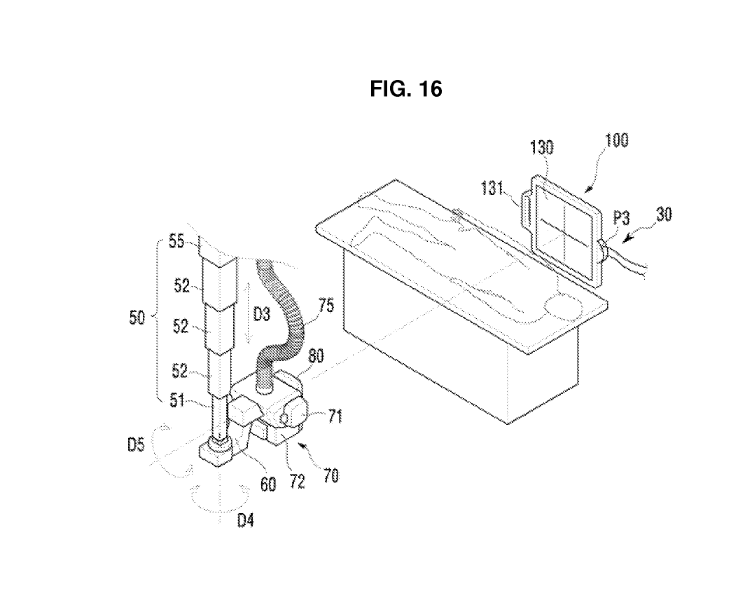

If the location determiner 310 determines that the X-ray detector 100 has been installed in the radiography portable 30, the user may set a radiography mode to “portable”. According to the user's setting, the motor controller 340 may output a control signal for controlling driving of the motor unit 110 in order to move the X-ray source 70 to the radiography portable 30. If the motor unit 110 is driven according to the control signal, the X-ray source 10 moves to the location of the X-ray detector 100 installed in the radiography portable 30, as illustrated in FIG. 16. In this state, X-rays irradiated from the X-ray source 70 can be detected by the X-ray detector 100 installed in the radiography portable 30.

Unlike the cases shown in FIGS. 14, 15, and 16, a case in which there are a plurality of radiography tables 10, a case in which there are a plurality of radiography stands 20, or a case in which there are a plurality of radiography portable 30 can be considered. For example, a case in which a first radiography table, a second radiography table, a first radiography stand, a second radiography stand, and a first radiography portable can be considered. In this case, if the location determiner 310 determines that the X-ray detector 100 has been installed in the first radiography table, the user may set a radiography mode to “first table”. According to the user's setting, the motor controller 340 may output a control signal(s) to a motor(s) that needs to be driven, and the motor unit 110 is driven according to the control signal to move the X-ray source 10 to correspond to the location of the X-ray detector 100 installed in the first radiography table.

The location determiner 310, the detector IP setting unit 320, and the motor controller 340 may be installed in one unit or in different units. For example, all of the location determiner 310, the detector IP setting unit 320, and the motor controller 340 may be installed in the workstation 170 (see FIG. 1). As an example, the location determiner 310 and the detector IP setting unit 320 may be installed in the workstation 170, and the motor controller 340 may be installed in the carriage 45 (see FIG. 1).

The X-ray imaging apparatus 1 may further include a storage unit, and the storage unit may store data or algorithms for manipulating the X-ray imaging apparatus 1.

The storage unit may store a digital value output from the ADC port 180 (see FIG. 4). Referring again to FIG. 12, the storage unit may store a value of 255 when the X-ray detector 100 has been installed in the radiography table 10, a value of 170 when the X-ray detector 100 has been installed in the radiography stand 20, and a value of 127 when the X-ray detector 100 has been installed in the radiography portable 30. As an example, the storage unit may store IP addresses in correspondence to the number of modules. Referring again to FIG. 13, the storage unit may store 192.168.0.1 set to an IP address for a radiography table, 192.168.0.2 set to an IP address for a radiography stand, and 192.168.0.3 set to an IP address for a radiography portable.

Also, the storage unit may store an algorithm for converting a monitored voltage into a digital value, and an algorithm for determining an installation location of the X-ray detector 100 based on the digital value.

The storage unit may be Read Only Memory (ROM), Programmable Read Only Memory (PROM), Erasable Programmable Read Only Memory (EPROM), non-volatile memory such as flash memory, volatile memory such as Random Access Memory (RAM), a hard disk, or an optical disk. However, the storage unit is not limited to the above-mentioned devices, and may be any other device well-known in the art.

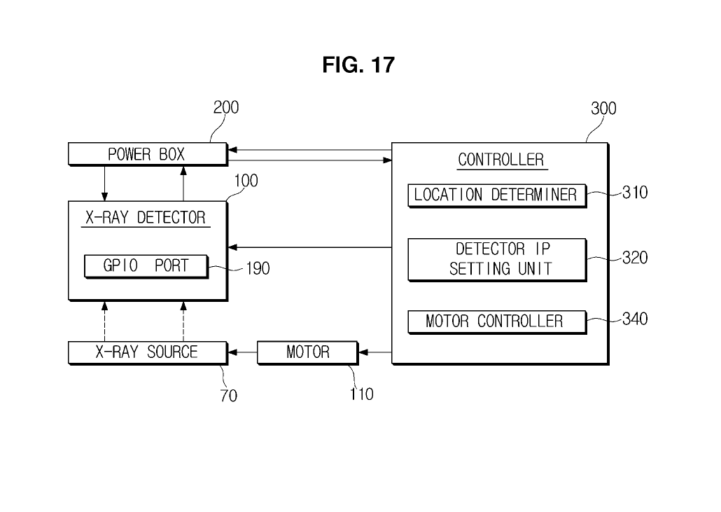

FIG. 17 is a control block diagram of an X-ray imaging apparatus according to an embodiment of the present disclosure.

Referring to FIG. 17, an X-ray imaging apparatus 1 may determine a location of an X-ray detector 100 using an X-ray source 70, the X-ray detector 100, a power box unit 200, a controller 300, and a motor unit 110, and move the X-ray source 70 to correspond to the location of the X-ray detector 100.

The X-ray source 70 and the motor unit 110 are the same as the X-ray source 70 and the motor unit 110 described above in the embodiment of FIG. 4, and accordingly, further descriptions thereof will be omitted. Also, in the following descriptions about the X-ray detector 100, the power box unit 200, and the controller 300, the same parts are described above with reference to FIG. 4, and descriptions thereof will be omitted.

The controller 300 may include a location determiner 310, a detector IP setting unit 320, and a motor controller 340.

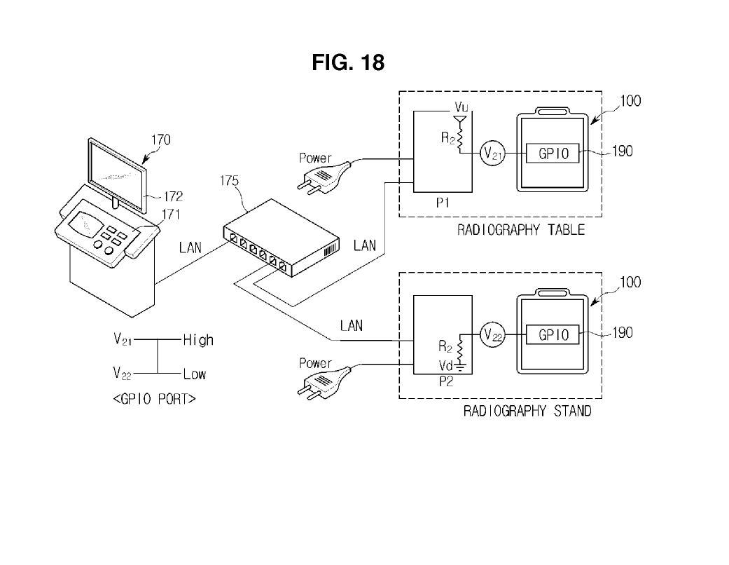

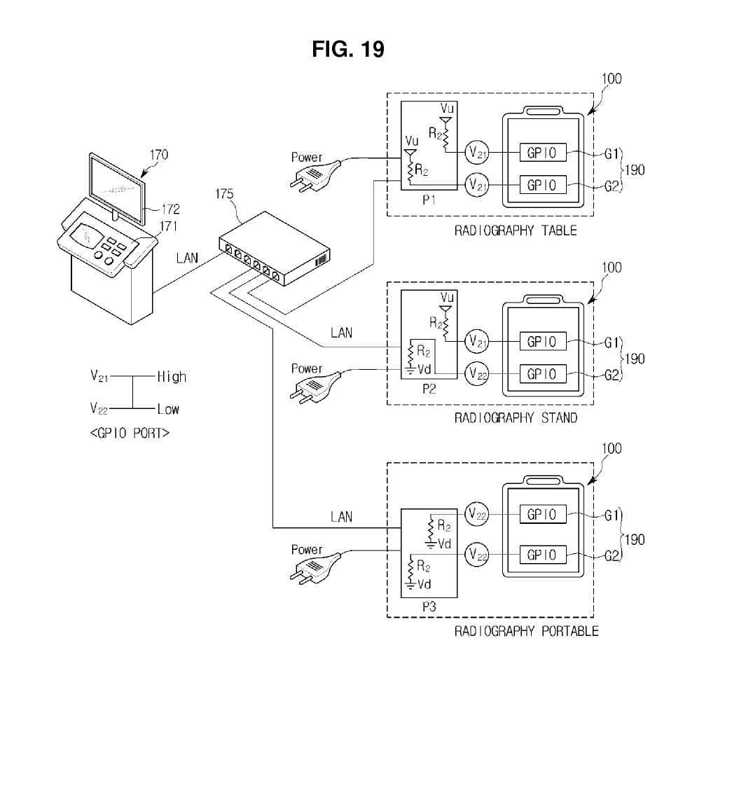

The location determiner 310 may determine an installation location of the X-ray detector 100. In order to help a determination of the location determiner 310, the X-ray detector 100 may include a port 190 (e.g., a General Purpose Input/Output (GPIO) port) for monitoring a voltage, and first, second, and third power boxes P1, P2, and P3 included in the power box unit 200 include unique ID resistors. A method in which the location determiner 310 determines an installation location of the X-ray detector 100 using the port and the ID resistors will be described in detail with reference to FIGS. 18 and 19, below.

FIG. 18 is a view for describing an example of a method of determining an installation location of the X-ray detector 100 based on the control block diagram of FIG. 17. In FIG. 18, a case in which there is a radiography table 10 and a radiography stand 20, and the location determiner 310 determines which one of the radiography table 10 and the radiography stand 20 the X-ray detector 100 has been installed in is shown.

The X-ray detector 100 may include a port for monitoring a voltage and outputting a relative level of the voltage. In FIG. 18, the port is a GPIO port 190. The first power box P1 includes a pull-up ID resistor R2, and the second power box P2 includes a pull-down ID resistor R2. The pull-up ID resistor R2 and the pull-down ID resistor R2 have the same resistance value of R2.

Because the ID resistor included in the first power box P1 is a pull-up resistor and the ID resistor included in the second power box P2 is a pull-down resistor although the ID resistors have the same resistance value, a value that the GPIO port 190 monitors may vary depending on which module the X-ray detector 100 has been installed in.

More specifically, if the X-ray detector 100 has been installed in the radiography table 10, the GPIO port 190 may monitor a voltage V21 applied to an ID check line in correspondence to the pull-up ID resistor R2 included in the first power box P1. If the X-ray detector 100 has been installed in the radiography stand 20, the GPIO port 190 may monitor a voltage V22 applied to an ID check line in correspondence to the pull-down ID resistor R2 included in the second power box P2. Because the ID resistor included in the first power box P1 is a pull-up resistor and the ID resistor included in the second power box P2 is a pull-down resistor, in other words, because a voltage Vu that is applied to the first power box P1 is higher than a voltage Vd that is applied to the second power box P2, the GPIO port 190 may monitor different voltages of V21 and V22 depending on an installation location of the X-ray detector 100. For example, if R2 is 5KΩ, Vu is 5V, and Vd is 0V, the voltage V21 becomes 5V and the voltage V22 becomes 0V.

The GPIO port 190 may output a relative level of a monitored voltage to the location determiner 310. Because a voltage that is monitored by the GPIO port 190 depends on which module the X-ray detector 100 has been installed in, a value that is output from the GPIO port 190 to the location determiner 310 also depends on which module the X-ray detector 110 has been installed in.

For example, if the X-ray detector 100 has been installed in the radiography table 10, and the GPIO port 190 has monitored a voltage V21 (e.g., 5V), the GPIO port 190 may output a high level corresponding to a relative level of 5V to the location determiner 310. If the X-ray detector 100 has been installed in the radiography stand 20, and the GPIO port 190 has monitored a voltage V22 (e.g., 0V), the GPIO port 190 may output a low level corresponding to a relative level of 0V to the location determiner 310.

Because the ID resistors have the same resistance value of R2, and a voltage that is applied to the X-ray detector 100 is constant in a module, the GPIO port 190 may output a constant value when the X-ray detector 100 has been installed in a module. If the X-ray detector 100 has been installed in the radiography table 10, the GPIO port 190 may output a high level, and if the X-ray detector 100 has been installed in the radiography stand 20, the GPIO port 190 may output a low level.

Accordingly, the location determiner 310 may determine an installation location of the X-ray detector 100 based on an output value from the GPIO port 190. If the GPIO port 190 has output a high level, the location determiner 310 may determine that the X-ray detector 100 has been installed in the radiography table 10, and if the GPIO port 190 has output a low level, the location determiner 310 may determine that the X-ray detector 100 has been installed in the radiography stand 20.

Meanwhile, if the X-ray imaging apparatus 1 further includes a storage unit, the storage unit may store an output value from the GPIO port 190 according to an installation location of the X-ray detector 100. In other words, the storage unit may store a high level when the X-ray detector 100 has been installed in the radiography table 10, and store a low level when the X-ray detector 100 has been installed in the radiography stand 20.

FIG. 19 is a view for describing an example of a method of determining an installation location of the X-ray detector 100 based on the control block diagram of FIG. 17. In FIG. 19, a case in which there is a radiography table 10 and a radiography stand 20, and the location determiner 310 determines which one of the radiography table 10 and the radiography stand 20 the X-ray detector 100 has been installed in is shown.

Referring to FIG. 19, the X-ray detector 100 may include two GPIO ports 190, that is, a first GPIO port G1 and a second GPIO port G2. Also, each of first, second, and third power boxes P1, P2, and P3 includes two ID resistors. More specifically, the first power box P1 includes two pull-up ID resistors, the second power box P2 includes a pull-up ID resistor and a pull-down ID resistor, and the third power box P3 includes two pull-down ID resistors. All of the pull-up ID resistors and the pull-down ID resistors have the same resistance value of R2. The ID resistors included in each of the first, second, and third power boxes P1, P2, and P3 can be expressed as an ordered pair according to an order in which the ID resistors are connected to the GPIO port 190. For example, the ID resistors included in the second power box P2 may be expressed as an ordered pair of a pull-up ID resistor R2 and a pull-down ID resistor R2.

The first GPIO port G1 and the second GPIO port G2 may monitor voltages applied to ID check lines, respectively.

More specifically, if the X-ray detector 100 has been installed in the radiography table 10, the first GPIO port G1 and the second GPIO port G2 may monitor a voltage V21 in correspondence to the pull-up ID resistors R2. If the X-ray detector 100 has been installed in the radiography stand 20, the first GPIO port G1 may monitor a voltage V21 in correspondence to the pull-down ID resistor R2, and the second GPIO port G2 may monitor a voltage V22 in correspondence to the pull-down ID resistor R2. If the X-ray detector 100 has been installed in the radiography portable 30, the first and second GPIO ports G1 and G2 may monitor a voltage V22 in correspondence to the pull-down ID resistors R2.

The first and second GPIO ports G1 and G2 may output relative levels of the monitored voltages to the location determiner 310.

For example, if the X-ray detector 100 has been installed in the radiography table 10 so that the first and second GPIO ports G1 and G2 have monitored a voltage V21 (e.g., 5V), the first and second GPIO ports G1 and G2 may output a high level corresponding to a relative level of 5V to the location determiner 310. If the X-ray detector 100 has been installed in the radiography stand 20 so that the first GPIO port G1 and the second GPIO port G2 have monitored a voltage V21 (e.g., 5V) and a voltage V22 (e.g., 0V), respectively, the first GPIO port G1 may output a high level corresponding to a relative level of 5V to the location determiner 310, and the second GPIO port G2 may output a low level corresponding to a relative level of 0V to the location determiner 310. If the X-ray detector 100 has been installed in the radiography portable 30 so that the first and second GPIO ports G1 and G2 have monitored a voltage V22 (e.g., 0V), the first and second GPIO ports G1 and G2 may output a low level corresponding to a relative level of 0V to the location determiner 310.

Accordingly, the location determiner 310 may determine an installation location of the X-ray detector 100 based on the output values from the first and second GPIO ports G1 and G2. If all of the first and second GPIO ports G1 and G2 have output a high level, the location determiner 310 may determine that the X-ray detector 100 has been installed in the radiography table 10, and if the first GPIO port G1 has output a high level and the second GPIO port G2 has output a low level, the location determiner 310 may determine that the X-ray detector 100 has been installed in the radiography stand 20. Also, if all of the first and second GPIO ports G1 and G2 have output a low level, the location determiner 310 may determine that the X-ray detector 100 has been installed in the radiography portable 30.

Meanwhile, the output values of the GPIO ports 190 may be expressed as an ordered pair according to an order of the GPIO ports 190. For example, if the X-ray detector 100 has been installed in the radiography stand 20 so that the first GPIO port G1 has output a high level and the second GPIO port G2 has output a low level, the output values of the GPIO ports 190 may be expressed as an ordered pair (High, Low).

If the X-ray imaging apparatus 1 further includes a storage unit, the storage unit may store output values of the first and second GPIO ports G1 and G2 according to an installation location of the X-ray detector 100. The storage unit may store a high level output from the first and second GPIO ports G1 and G2 when the X-ray detector 100 has been installed in the radiography table 10, a high level output from the first GPIO port G1 and a low level output from the second GPIO port G2 when the X-ray detector 100 has been installed in the radiography stand 20, and a low level output from the first and second GPIO ports G1 and G2 when the X-ray detector 100 has been installed in the radiography portable 30.

For convenience of description, a case in which the X-ray imaging apparatus 1 includes a radiography table 10 and a radiography stand 20, or a case in which the X-ray imaging apparatus 1 includes a radiography table 10, a radiography stand 20, and a radiography portable 30 has been described. However, the number of modules is not limited.

One or more GPIO ports 190 may be provided according to the number of modules. Also, one or more ID resistors may be provided according to the number of the GPIO ports 190 so that the ID resistors can one-to-one match the GPIO ports 190. The ID resistors have a different configuration for each power box unit 200 so that output values from the GPIO ports 190 vary depending on which module the X-ray detector 100 has been installed in.

Accordingly, even when the number of modules is greater or smaller than those illustrated in FIGS. 18 and 19, the location determiner 310 may determine an installation location of the X-ray detector 100 based on output values of the GPIO ports 190.

The detector IP setting unit 320 may set IP addresses in correspondence to the number of modules, and assign an IP address corresponding to an installation location of the X-ray detector 100 to the X-ray detector 100, or change an IP address assigned to the X-ray detector 100 according to an installation location of the X-ray detector 100.

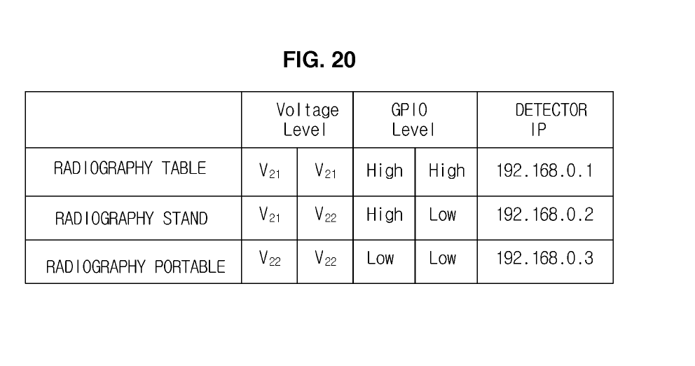

FIG. 20 is a view for describing a method of assigning an IP address to the X-ray detector 100 and a method of changing an IP address of the X-ray detector 100. In FIG. 20, an IP address for a radiography table 10 has been set to 192.168.0.1, an IP address for a radiography stand 20 has been set to 192.168.0.2, and an IP address for a radiography portable 30 has been set to 192.168.0.3, based on the example shown in FIG. 19.

As described above with reference to FIG. 19, if the first and second GPIO ports G1 and G2 have monitored a voltage V₂₁ and output a high level corresponding to a relative level of the voltage V₂₁, the location determiner 310 may determine that the X-ray detector 100 has been installed in the radiography table 10. Based on the determination of the location determiner 310, the detector IP setting unit 320 may assign the IP address 192.168.0.1 for the radiography table 10 to the X-ray detector 100. If an IP address that is different from 192.168.0.1 has been assigned to the X-ray detector 100, the detector IP setting unit 320 may change the IP address of the X-ray detector 100 to 192.168.0.1 so that the IP address of the X-ray detector 100 is identical to the IP address for the radiography table 10.

If the first GPIO port G1 has monitored a voltage V₁₂ and output a high level while the second GPIO port G2 has monitored a voltage V₂₂ and output a low level, the location determiner 310 may determine that the X-ray detector 100 has been installed in the radiography stand 20. Accordingly, the detector IP setting unit 320 may assign the IP address 192.168.0.2 for the radiography stand 20 to the X-ray detector 100. If an IP address that is different from 192.168.0.2 has been assigned to the X-ray detector 100, the detector IP setting unit 320 may change the IP address of the X-ray detector 100 to 192.168.0.2 so that the IP address of the X-ray detector 100 is identical to the IP address for the radiography stand 20.

If the first and second GPIO ports G1 and G2 have monitored a voltage V₂₂ and output a low level corresponding to a relative level of the voltage V₂₂, the location determiner 310 may determine that the X-ray detector 100 has been installed in the radiography portable 30. Accordingly, the detector IP setting unit 320 may assign the IP address 192.168.0.3 for the radiography portable 30 to the X-ray detector 100. If an IP address that is different from 192.168.0.3 has been assigned to the X-ray detector 100, the detector IP setting unit 320 may change the IP address of the X-ray detector 100 to 192.168.0.3 so that the IP address of the X-ray detector 100 is identical to the IP address for the radiography portable 30.

As such, because the detector IP setting unit 320 assigns an IP address to the X-ray detector 100 or changes an IP address of the X-ray detector 100 according to an installation location of the X-ray detector 100, the X-ray detector 100 can be used at various locations.

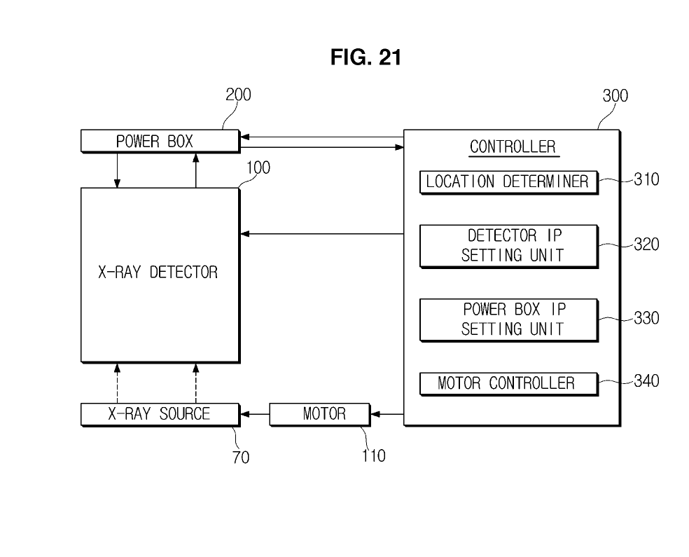

FIG. 21 is a control block diagram of an X-ray imaging apparatus according to an embodiment of the present disclosure.

Referring to FIG. 21, an X-ray imaging apparatus 1 may determine a location of the X-ray detector 100 using an X-ray source 70, the X-ray detector 100, a power box unit 200, a controller 300, and a motor unit 110, and move the X-ray source 70 to correspond to the location of the X-ray detector 100. The controller 300 may include a location determiner 310, a detector IP setting unit 320, a power box IP setting unit 330, and a motor controller 340.

The X-ray source 70, the X-ray detector 100, the motor unit 110, and the motor controller 340 are the same components as those used in the above-described embodiments, and accordingly, further descriptions thereof will be omitted. Also, in the following descriptions about the power box unit 200, the location determiner 310, and the detector IP setting unit 320, the same parts as those described in the above embodiments will be omitted.

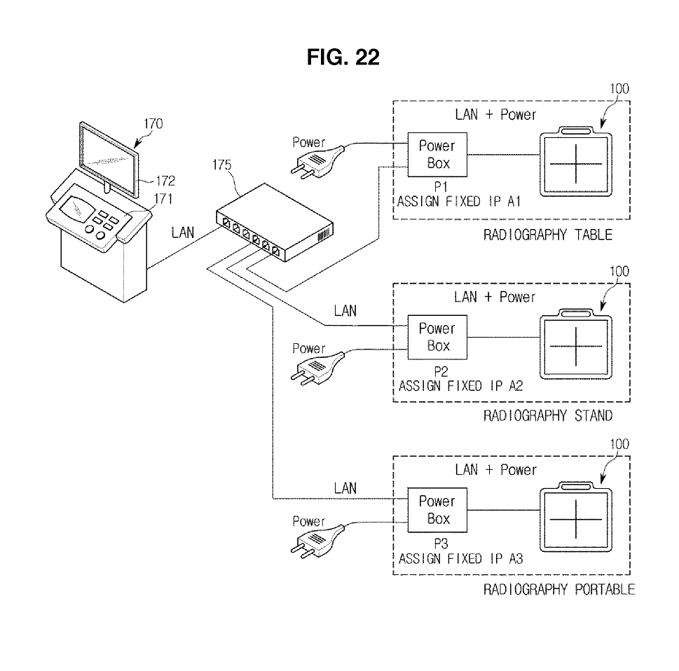

FIG. 22 is a view for describing an example of a method of determining an installation location of the X-ray detector 100 based on the control block diagram of FIG. 21. In the example of FIG. 22, a radiography table 10, a radiography stand 20, and a radiography portable 30 are provided. Accordingly, the location determiner 310 may determine which one of the radiography table 10, the radiography stand 20, and the radiography portable 30 the X-ray detector 100 has been installed in.

Referring to FIG. 22, the power box IP setting unit 330 may set IP addresses in correspondence to the number of modules, and assign the IP addresses, respectively, to first, second, and third power boxes P1, P2, and P3 included in the power box unit 200. The IP addresses assigned to the first, second, and third power boxes P1, P2, and P3 do not change. That is, the power box IP setting unit 330 may assign static IP addresses to the first, second, and third power boxes P1, P2, and P3, respectively.

More specifically, the power box IP setting unit 330 may set a static IP address for the radiography table 10 to A1, a static IP address for the radiography stand 20 to A2, and a static IP address for the radiography portable 30 to A3, respectively, in correspondence to the number of modules. Then, the power box IP setting unit 330 may assign the static IP address A1 to the first power box P1, the static IP address A2 to the second power box P1, and the static IP address A3 to the third power box P3. The static IP addresses A1, A2, and A3 are different values.

The detector IP setting unit 320 may set IP addresses in correspondence to the number of modules, assign an IP address corresponding to an installation location of the X-ray detector 100 to the X-ray detector 100, and maintain or change an IP address of the X-ray detector 100 according to an installation location of the X-ray detector 100.

First, the detector IP setting unit 320 may set IP addresses in correspondence to the number of modules, and assign an IP address to the X-ray detector 100. More specifically, the detector IP setting unit 320 may set an IP address for the radiography table 10 to B1, an IP address for the radiography stand 20 to B2, and an IP address for the radiography portable 30 to B3, in correspondence to the number of modules. Then, the detector IP setting unit 320 may assign one of the IP addresses to the X-ray detector 100. That is, an IP address that is assigned to the X-ray detector 100 may be one of B1, B2, and B3.

Thereafter, the location determiner 310 determines an installation location of the X-ray detector 100. At this time, the static IP addresses assigned to the first, second, and third power boxes P1, P2, and P3 are used. More specifically, before determining an installation location of the X-ray detector 100, the location determiner 310 may request an IP address of the X-ray detector 100 and a static IP address of a power box P1, P2 or P3 connected to the X-ray detector 100. For example, if an IP address assigned to the X-ray detector 100 is B2, and a static IP address of a power box connected to the X-ray detector 100 is A1, the location determiner 310 may determine that the X-ray detector 100 for the radiography stand 20 has been connected to the first power box P1, that is, that the X-ray detector 100 has been installed in the radiography table 10.

The detector IP setting unit 320 may maintain or change an IP address of the X-ray detector 100 according to the installation location of the X-ray detector 100. Because the X-ray detector 100 allocated an IP address B2, that is, the X-ray detector 100 for the radiography stand 20 has been installed in the radiography table 10, the detector IP setting unit 320 may change the IP address of the X-ray detector 100 from B2 to B1 to thus convert the X-ray detector 100 for the radiography stand 20 to the X-ray detector 100 for the radiography table 10.

Meanwhile, if the X-ray imaging apparatus 1 further includes a storage unit, the storage unit may store the IP addresses for the power boxes and the IP addresses for the X-ray detector 100, set in correspondence to the number of modules. Referring again to FIG. 22, the storage unit may store A1 set to an IP address for the radiography table 10, A2 set to an IP address for the radiography stand 20, and A3 set to an IP address for the radiography portable 30 so that the IP addresses A1, A2, and A3 can be assigned to the power boxes. Also, the storage unit may store B1 set to an IP address for the radiography table 10, B2 set to an IP address for the radiography stand 20, and B3 set to an IP address for the radiography portable 30 so that the IP address B1, B2, or B3 can be assigned to the X-ray detector 100.

The storage unit may store algorithms for determining an installation location of the X-ray detector 100 using a static IP address of a power box.

In FIGS. 21 and 22, for convenience of description, a case in which there are a radiography table 10, a radiography stand 20, and a radiography portable 30 has been described. However, the number of modules is not limited.

Components of the X-ray imaging apparatuses according to the above-described embodiments, and functions of the components have been described. Hereinafter, a control method of the X-ray imaging apparatus 1 will be described with reference to a given flowchart. In the following description, for convenience of description, it is assumed that the X-ray imaging apparatus 1 includes a radiography table 10, a radiography stand 20, and a radiography portable 30.

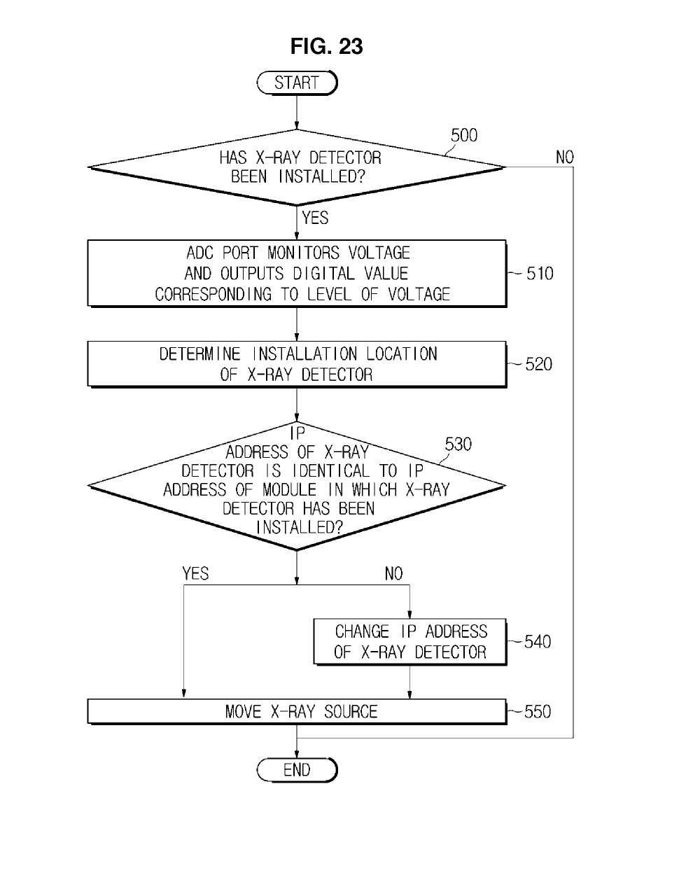

FIG. 23 is a flowchart illustrating a control method of an X-ray imaging apparatus, according to an embodiment of the present disclosure.

In FIG. 23, the power boxes (the first power box P1, the second power box P2, and the third power box P3) of the radiography table 10, the radiography stand 20, and the radiography portable 30 (see FIG. 22) include different ID resistors.

Referring to FIGS. 4, 12, and 23, the workstation 170 may determine whether the X-ray detector 100 has been installed (operation 500).

More specifically, if the workstation 170 requests individual modules to determine whether the X-ray detector 100 has been installed therein, the modules wait for an acknowledgement from the X-ray detector 100. If a module that has received an acknowledgement from the X-ray detector 100 is found, the workstation 170 may determine that the X-ray detector 100 has been installed.

If no module that has received an acknowledgement from the X-ray detector 100 is found, the process terminates.

If the workstation 170 determines that the X-ray detector 100 has been installed, the ADC port 180 of the X-ray detector 100 may monitor a voltage applied to an ID check line, convert the monitored voltage into a digital value corresponding to a level of the voltage, and output the digital value to the location determiner 310 (operation 510).

The location determiner 310 may determine an installation location of the X-ray detector 100, based on the digital value output from the ADC port 180 (operation 520).

Because ID resistors included in the power boxes have different resistance values, a voltage that is monitored by the ADC port 180 varies depending on which module the X-ray detector 100 has been installed in. In other words, a value which the ADC port 180 outputs to the location determiner 310 varies depending on which module the X-ray detector 100 has been installed in. Accordingly, the location determiner 310 may determine an installation location of the X-ray detector 100 based on the digital value output from the ADC port 180.

The detector IP setting unit 320 may assign an IP address of a module in which the X-ray detector 100 has been installed, to the X-ray detector 100. If the X-ray detector 100 has already been assigned an IP address, the detector IP setting unit 320 may determine whether the IP address assigned to the X-ray detector 100 is identical to the IP address of the module in which the X-ray detector 100 has been installed (operation 530).

If the detector IP setting unit 320 determines that the IP address assigned to the X-ray detector 100 is not identical to the IP address of the module in which the X-ray detector 100 has been installed, the detector IP setting unit 320 may change the IP address of the X-ray detector 100 to the IP address of the module in which the X-ray detector 100 has been installed (operation 540).

If the detector IP setting unit 320 determines that the IP address assigned to the X-ray detector 100 is identical to the IP address of the module in which the X-ray detector 100 has been installed, or if the detector IP setting unit 320 changes the IP address of the X-ray detector 100 to the IP address of the module in which the X-ray detector 100 has been installed, the X-ray source 100 may move to correspond to the location of the X-ray detector 100 (operation 550).

More specifically, in a manual move mode, a user may apply power or torque to the handle 82 of the operating unit 80 (see FIG. 3) to move the X-ray source 70. In an automatic move mode, the user may set a radiography mode corresponding to an installation location of the X-ray detector 100, and according to the user's setting, the motor unit 110 may be driven to move the X-ray source 70.

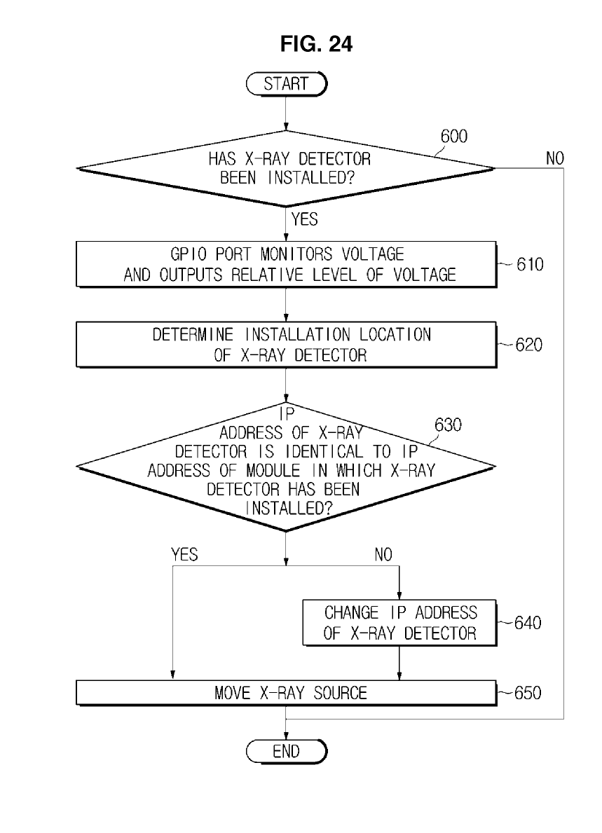

FIG. 24 is a flowchart illustrating a control method of an X-ray imaging apparatus, according to an embodiment of the present disclosure.

In the following description, descriptions of the same parts as in the above-described embodiment will be omitted.

In FIG. 24, it is assumed that the X-ray detector 100 includes two GPIO ports 190, that is, a first GPIO port G1 and a second GPIO port G2, and each of power boxes P1, P2, and P3 includes two ID resistors. For example, as illustrated in FIG. 19, the first power box P1 includes two pull-up ID resistors that can be respectively connected to the first and second GPIO ports G1 and G2, the second power box P2 includes a pull-up ID resistor that can be connected to the first GPIO port G1 and a pull-down ID resistor that can be connected to the second GPIO port G2, and the third power box P3 includes two pull-down ID resistors that can be respectively connected to the first and second GPIO ports G1 and G2. The pull-up ID resistors and the pull-down ID resistors have the same resistance value. That is, the ID resistors included in the first power box P1 can be expressed as an ordered pair of two pull-up ID resistors R₂, the ID resistors included in the second power box P2 can be expressed as an ordered pair of a pull-up ID resistor R₂ and a pull-down ID resistor R₂, and the ID resistors included in the third power box P3 can be expressed as an ordered pair of two pull-down ID resistors R₂.

Referring to FIGS. 17, 19, and 24, the workstation 170 may determine whether the X-ray detector 100 has been installed (operation 600).

If the workstation 170 determines that no X-ray detector 100 has been installed, the process terminates.

If the workstation 170 determines that the X-ray detector 100 has been installed, the first GPIO port G1 and the second GPIO port G2 may monitor voltages applied to ID check lines, respectively. Then, the first GPIO port G1 and the second GPIO port G2 may output relative levels of the monitored voltages, respectively, to the location determiner 310 (operation 610).

The location determiner 310 may determine an installation location of the X-ray detector 100, based on the output values from the first and second GPIO ports G1 and G2 or an ordered pair of the output values from the first and second GPIO ports G1 and G2 (operation 620).

Because the ID resistors have a different configuration for each power box, voltages that are monitored by the first and second GPIO ports G1 and G2 vary depending on an installation location of the X-ray detector 100. In other words, values that are output from the first and second GPIO ports G1 and G2 to the location determiner 310, or an ordered pair of the output values from the first and second GPIO ports GI and G2 may vary depending on which module the X-ray detector 100 has been installed in. Accordingly, the location determiner 310 may determine an installation location of the X-ray detector 100, based on output values of the first and second GPIO ports G1 and G2 or an ordered pair of output values of the first and second ports G1 and G2.

The detector IP setting unit 320 may assign an IP address of a module in which the X-ray detector 100 has been installed, to the X-ray detector 100. If the X-ray detector 100 has already been assigned an IP address, the detector IP setting unit 320 may determine whether the IP address assigned to the X-ray detector 100 is identical to the IP address of the module in which the X-ray detector 100 has been installed (operation 630).

If the detector IP setting unit 320 determines that the IP address assigned to the X-ray detector 100 is not identical to the IP address of the module in which the X-ray detector 100 has been installed, the detector IP setting unit 320 may change the IP address of the X-ray detector 100 to the IP address of the module (operation 640).

If the detector IP setting unit 320 determines that the IP address assigned to the X-ray detector 100 is identical to the IP address of the module, or if the detector IP setting unit 320 changes the IP address of the X-ray detector 100 to the IP address of the module, the X-ray source 70 may move to correspond to the location of the X-ray detector 100 (operation 650).

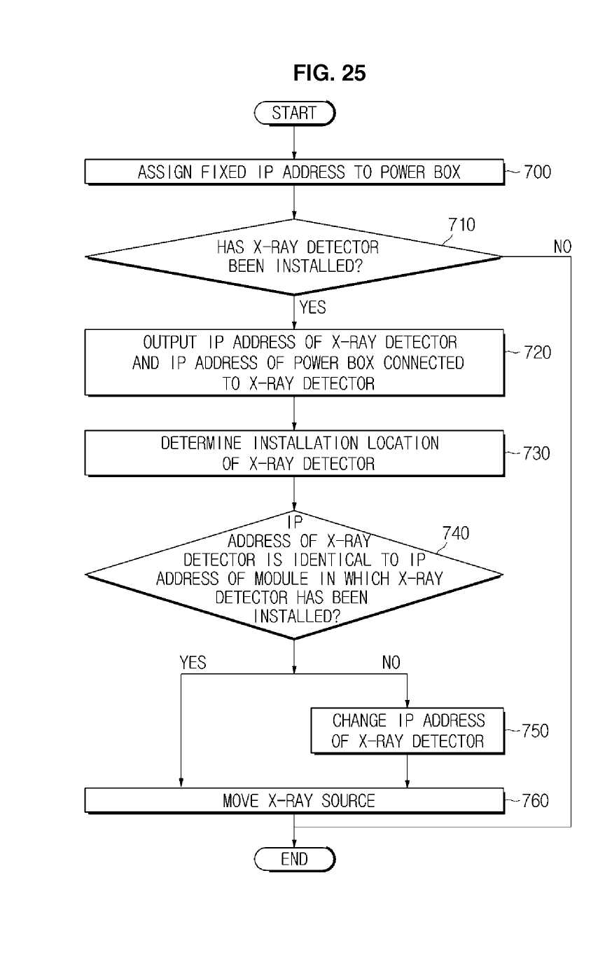

FIG. 25 is a flowchart illustrating a control method of an X-ray imaging apparatus, according to an embodiment of the present disclosure. In the following description, descriptions of the same parts as in the above-described embodiment will be omitted.

Referring to FIGS. 21, 22, and 25, first, the detector IP setting unit 320 may assign an IP address to the X-ray detector 100. Then, the power box IP setting unit 330 may assign static IP addresses to the first, second, and third power boxes P1, P2, and P3 (operation 700). That is, IP addresses assigned to the first, second, and third power boxes P1, P2, and P3 do not change. Also, the static IP addresses respectively assigned to the first, second, and third power boxes P1, P2, and P3 are different values.

After the static IP addresses are assigned to the first, second, and third power boxes P1, P2, and P3, the workstation 170 may determine whether the X-ray detector has been installed (operation 710).

More specifically, if the workstation 170 requests individual modules to determine whether the X-ray detector 100 has been installed therein, the modules wait acknowledge from the X-ray detector 100. If a module that has received acknowledge from the X-ray detector 100 is found, the workstation 170 may determine that the X-ray detector 100 has been installed. The module that has received acknowledge from the X-ray detector 100 is referred to as a module M.

If the workstation 170 determines that no X-ray detector 100 has been installed, the process terminates.

If the workstation 170 determines that the X-ray detector 100 has been installed, the module M may output an IP address of the X-ray detector 100 and a static IP address of a power box connected to the X-ray detector 100 (operation 720). More specifically, the module M that has received acknowledge from the X-ray detector 100 may request the X-ray detector 100 to send an IP address, and receive an IP address from the X-ray detector 100. Then, the module M may transmit the IP address of the X-ray detector 100 and the static IP address of the power box connected to the X-ray detector 100, that is, the static IP address of the power box included in the module M to the workstation 170.

The location determiner 310 may determine an installation location of the X-ray detector 100 based on the IP addresses received from the module M (operation 730).

For example, if the IP address of the X-ray detector 100 is an IP address for the radiography stand 20, and the static IP address of the power box is an IP address for the radiography table 10, the location determiner 310 may determine that the X-ray detector 100 has been installed in the radiography table 10.

Thereafter, the detector IP setting unit 320 may determine whether the IP address of the X-ray detector 100 is identical to an IP address of a module in which the X-ray detector 100 has been installed (operation 740).

If the detector IP setting unit 320 determines that the IP address of the X-ray detector 100 is not identical to the IP address of the module, the detector IP setting unit 320 may change the IP address of the X-ray detector 100 to the IP address of the module (operation 750). Because the IP address of the X-ray detector 100 is an IP address for the radiography stand 20, and a module in which the X-ray detector 100 has been installed is the radiography table 10, the detector IP setting unit 320 may change the IP address of the X-ray detector 100 to the IP address for the radiography table 10 so that the X-ray detector 100 can detect X-rays in the radiography table 10.

If the detector IP setting unit 320 determines that the IP address of the X-ray detector 100 is identical to the IP address of the module, or if the detector IP setting unit 320 changes the IP address of the X-ray detector 100 to the IP address for the radiography table 10, the X-ray source 70 may move to correspond to the location of the X-ray detector 100 (operation 760).

The above-described embodiments may be recorded in computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter. The above-described devices may be configured to act as one or more software modules in order to perform the operations of the above-described embodiments, or vice versa.

Although a few embodiments of the present disclosure have been shown and described, it would be appreciated by those skilled in the art that changes may be made in these embodiments without departing from the principles and spirit of the disclosure, the scope of which is defined in the claims and their equivalents.

What is claimed is:

1. An X-ray imaging apparatus comprising:
an X-ray source configured to generate and irradiate X-rays;
an X-ray detector installed in a module of a plurality of modules, and configured to detect the irradiated X-rays;
an Identification (ID) resistor comprised in the module;
a port comprised in the X-ray detector; and
a controller configured to determine the module in which the X-ray detector has been installed, using the ID resistor and the port.

2. The X-ray imaging apparatus according to claim 1, wherein the module comprises at least one radiography table.

3. The X-ray imaging apparatus according to claim 1, wherein the module comprises at least one radiography stand.

4. The X-ray imaging apparatus according to claim 1, wherein the module comprises at least one radiography portable.

5. The X-ray imaging apparatus according to claim 1, wherein each module comprises a power box connected to the controller and a power supply unit.

6. The X-ray imaging apparatus according to claim 5, wherein the ID resistor is comprised in the power box.

7. The X-ray imaging apparatus according to claim 5, wherein the power box of the module in which the X-ray detector has been installed is connected to the X-ray detector so that power is supplied to the X-ray detector and the X-ray detector is able to communicate with the controller.

8. The X-ray imaging apparatus according to claim 1, wherein the port includes an Analog-Digital Converter (ADC) port.

9. The X-ray imaging apparatus according to claim 1, wherein ID resistors having different resistance values are respectively included in different power boxes.

10. The X-ray imaging apparatus according to claim 5, wherein the port monitors a voltage in correspondence to the ID resistor of the power box connected to the X-ray detector, and outputs a digital value corresponding to a level of the voltage to the controller.

11. The X-ray imaging apparatus according to claim 1, wherein the controller determines the module in which the X-ray detector has been installed, based on a digital value output from the port.

12. The X-ray imaging apparatus according to claim 1, wherein the port includes a General Purpose Input/Output (GPIO) port.

13. The X-ray imaging apparatus according to claim 1, wherein the port is at least one port included in the X-ray detector.

14. The X-ray imaging apparatus according to claim 13, wherein the ID resistor is at least one ID resistor included in the power box in correspondence with the number of the at least one port.

15. The X-ray imaging apparatus according to claim 14, wherein the at least one port one-to-one matches the at least one ID resistor included in the power box.

16. The X-ray imaging apparatus according to claim 15, wherein each of the at least one ID resistor has the same resistance value.

17. The X-ray imaging apparatus according to claim 16, wherein each of the at least one ID resistor is a pull-up resistor or a pull-down resistor.

18. The X-ray imaging apparatus according to claim 17, wherein different ordered pairs of ID resistors are respectively included in different power boxes.

19. The X-ray imaging apparatus according to claim 18, wherein each of the at least one port monitors a voltage in correspondence to an ID resistor matching the corresponding port among at least one ID resistor of a power box to which the X-ray detector has been connected, and outputs a relative level of the voltage to the controller.

20. The X-ray imaging apparatus according to claim 19, wherein the controller determines the module in which the X-ray detector has been installed, based on the relative level of the voltage output from the port.

21. An X-ray imaging apparatus comprising:
an X-ray source configured to generate and irradiate X-rays;
an X-ray detector installed in a module of a plurality of modules, and configured to detect the irradiated X-rays; and
a controller configured to assign a static Information Provider (IP) address to the module, and to determine the module in which the X-ray detector has been installed, based on the static IP address.

22. The X-ray imaging apparatus according to claim 21, wherein each module comprises a power box connected to the controller and a power supply unit.

23. The X-ray imaging apparatus according to claim 22, wherein the static IP address is assigned to the power box.

24. The X-ray imaging apparatus according to claim 22, wherein the power box of the module in which the X-ray detector has been installed is connected to the X-ray detector so that power is supplied to the X-ray detector and the X-ray detector is able to communicate with the controller.

25. The X-ray imaging apparatus according to claim 22, wherein the controller receives the static IP address of the power box to which the X-ray detector is connected.

26. The X-ray imaging apparatus according to claim 22, wherein the controller determines the module in which the X-ray detector has been installed, based on the static IP address.

27. The X-ray imaging apparatus according to claim 21, wherein the controller maintains or changes the IP address assigned to the X-ray detector in correspondence with the module in which the X-ray detector has been installed.

28. The X-ray imaging apparatus according to claim 21, wherein the controller moves the X-ray source to correspond to a location of the X-ray detector.

29. A control method of an X-ray imaging apparatus, the method comprising:
monitoring, at a port included in an X-ray detector, a voltage in correspondence with an Identification (ID) resistor of a module in which the X-ray detector has been installed;
outputting, from the port, a data value corresponding to the voltage; and
determining the module in which the X-ray detector has been installed, based on the data value.

30. A control method of an X-ray imaging apparatus, the method comprising:
assigning a static Information Provider (IP) address to a module;
receiving a static IP address of the module in which an X-ray detector has been installed; and
determining the module in which an X-ray detector has been installed, based on the received static IP address.

31. The control method according to claim 30, further comprising maintaining or changing an IP address assigned to the X-ray detector in correspondence to the module in which the X-ray detector has been installed.

32. The control method according to claim 30, further comprising moving the X-ray source to correspond to a location of the X-ray detector.

33. An X-ray imaging system comprising:
a first X-ray station comprising a first identifier having a first value;
a second X-ray station comprising a second identifier having a second value different from the first value;
an X-ray detector configured to be installed in the first X-ray station or the second X-ray station;
a port configured to detect either the first value or the second value; and
a controller configured to determine whether the X-ray detector is installed in the first X-ray station or the second X-ray station based on the detected value.

34. The X-ray imaging system of claim 33, further comprising:
an X-ray source,
wherein the controller is configured to move the X-ray source to the X-ray station where the X-ray detector was determined to be installed.

35. The X-ray imaging system of claim 33,
wherein the first identifier comprises a first identification resistor,
the first value comprises a first resistance value,
the second identifier comprises a second identification resistor, and
the second value comprises a second resistance value,

36. The X-ray imaging system of claim 33,
wherein the first identifier comprises a first Information Provider address,
the first value comprises the first Information Provider address,
the second identifier comprises a second Information Provider address, and
the second value comprises the second Information Provider address.
